SEQUENCE LISTING (28 sequences)

SEQ ID NO: 1 - protein, 6 aa, Artificial Sequence
  Synthetic peptide-like compound
XXXXXX

SEQ ID NO: 2 - protein, 6 aa, Artificial Sequence
  Synthetic peptide-like compound
XXAAXK

SEQ ID NO: 3 - protein, 6 aa, Artificial Sequence
  Synthetic peptide-like compound
AXAWXK

SEQ ID NO: 4 - protein, 6 aa, Artificial Sequence
  Synthetic peptide-like compound
HXAWXA

SEQ ID NO: 5 - protein, 6 aa, Artificial Sequence
  Synthetic peptide-like compound
AXAWXK

SEQ ID NO: 6 - protein, 6 aa, Artificial Sequence
  Synthetic peptide-like compound
HXXWAK

SEQ ID NO: 7 - protein, 6 aa, Artificial Sequence
  Synthetic peptide-like compound
HXXAXK

SEQ ID NO: 8 - protein, 6 aa, Artificial Sequence
  Synthetic peptide-like compound
FXAXXK

SEQ ID NO: 9 - protein, 6 aa, Artificial Sequence
  Synthetic peptide-like compound
AXAXXK

SEQ ID NO: 10 - protein, 6 aa, Artificial Sequence
  Synthetic peptide-like compound
XXAXXK

SEQ ID NO: 11 - protein, 6 aa, Artificial Sequence
  Synthetic peptide-like compound
AXXWXK

SEQ ID NO: 12 - protein, 6 aa, Artificial Sequence
  Synthetic peptide-like compound
AXAXXK

SEQ ID NO: 13 - protein, 6 aa, Artificial Sequence
  Synthetic peptide-like compound
HXXWXK

SEQ ID NO: 14 - protein, 6 aa, Artificial Sequence
  Synthetic peptide-like compound
HXXWXA

SEQ ID NO: 15 - protein, 6 aa, Artificial Sequence
  Synthetic peptide-like compound
XXXWXK

SEQ ID NO: 16 - protein, 6 aa, Artificial Sequence
  Synthetic peptide-like compound
XXXWXK

SEQ ID NO: 17 - DNA, 4727 bp, Homo sapiens
  CDS (415)..(1833)
ctttcaattcctctggcaacaaaccacacactgggatctgacactgtagagtgctttctcttctctttttttgggggggggagggggtgtggttgcatatttaaactctcacgcatttatgtactgaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataaacctggctcaagcacaaaccaatttgtgttgttctgattcaataattggtttctgggtggccaattcagaagaagagtgtacatgctcaacaaatcctaggccctgcattcctgtcatcctcatccgggggaaacaccatcatcccagtagctgccctattcaactgcaacagtctccaggaccatcagtatactgcatttcatgtgcaccaaatattttgaaagacatttataaataattggcttatgactcatatttctctatgaataccttcatacagcaggtataactcttttctttatgggcttaaatattttgtcactgatcctgcaaatggacatcattttagcacactagcggtttatattttaaggaccttcattctctgttctgcacctcttctggaaattgagtaaattttgcttttttttttttactcagttgcaacttacgcttggcatcttcagaatgcttttctagcattaagagatgtaaatgataaaggaattattgtatgaaatattacaaagcgtagactatgcattgttattcattataatattttttgctgtcataatcgcctcataaagacaggtttcaaccattaaaatatgttcttccttaaattcctgtgctttttctagttcctcttgtgtcataaaatgtttatcctaattttctctctgaagtatattttatctgaatccacatttctttataaatccatagtccttgctgaaatatgctttctaaatttctaccactttgttctaggctaattttttaagctaattggatgaagaacaaaaagacatttggtttcatcctttacagcagtaggacaattgcaaaggtttttcctttttcataaggagacacattaataggtaactctgtttcttgagcaggggttcacttattctgagagcattagttctcctaaaaagctccagcatagaaagggaagataaaccaaattctagcttgtgttttacccacagaaggatacaggacaaaggaatagtaactggcctgtttggatactaaaatcgaaaataacttttagcctcctccttatgatagccgccagagtaaatgttgagcattactacagaaaagccacaaaccaagaatctacctgtttggaaagatcttttgcatctctgaaggtgcttaaagcatacttagtgcctttccttttaactgggaagataaaagaagtatctgtccaagatattaatatgtaagataacattgtagacatgttcttctgataatacaaggtttattctatttgcattaggatatttgtggacatgtccatctaatataaaggaaagttttttaatcattgaggcatgtagggctgagttatataatgtagaaacttctaaagataattggatgagaatatacatattgacctgtatattatgactaatcatgactcagatcttaatacagggatgatctcatagcatttagatatcagaaaaggttttgacctatatgtctttaatattgtttgaatacatgtataatctttatcattcctcagtgtttcatttctcaaattctgtaaaaggaatataagaggaaagacaattcatatacaaagacaacgagattaaaaatatgcagtaggaaaaataattacttaaggggagattttttttacatgaaatctgggctttggatgtgtgtgtgtgtgtgtgtgtgtgtgtgtgtgcacatatgcactgtggtgggagtggggcaacttggggaatatgttacatgtgtgactttgttttgccctggcgaagttaatgttgttcagaaagggtaaatgtttggacacttgcaattgctcatggatgaatttatatgttttagtcatagaaaaattgtaccctttgatagaagcacattttctttccaaagctggttattaaccacagaattatagcaggtattcataacttaagtttgaaaatcaatagcgtctgcaaatggattaacagattagagaatcaacagcatcggaaaataggttaatgcatattgcttctaacaagtgcatgaagaaatagaagaagctatgtagctttcagttctgacagaaaagggtgaaggagggtatcatttcaagaaaaaaaatagctatcacgcaatggttatctctgaaaatatttgtattaagatgtgtatacatggccaggcatggtggctcatgcctgtaatcccagcactttgggaggcaggtggatcacgaggtcaggagatcaagaccatcctggccaacatggtgaaacctcatctctactaaaaatacaaaaatgagcggggtgtggtggcccatgcctgtagtcccagctgctcgggagactgaatctcttgagcctgggaagcagaggttgcagtgaactgagatcgcgtcactgcactccagcctggtgacagagcgagattccatctcaaaaaaaaaaaacagtatgcacgtacaaatttcttaacctgttatcaatgtctgagctacataattatctttctagttggagtttgttttaggtgtgtaccaactgacatttcagtttttctgtttgaagtccaatgtattagtgactctgtggctgctctcttcacctgccccttgtggcctgtctacaattctaaatggattttgaactcaatgtcgtcgcttctggtttcctgcatataccaatagcattacctatgactttttttttcctgagctattttcactgagctgagctaatgaactaaaactgagttatgtttaatatttgtatcaaatacataaaaggaatactgctttttccttttgtggctcaaaggtagctgcattttaaaatatttgtgaaaataaaaacttttgttattagaaaaatga

SEQ ID NO: 18 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 19 - DNA, 2069 bp, Homo sapiens
  CDS (376)..(1794)
gaggatgtcaatggctttcagatgtcaggataaccttaaggatagatgaagggttgagagcctgtgcctcatttctgagttctcagctgctatgccgtggaaatcctgtttactttctgcatctgctcctgcaagactctggagccagtcttgaggtcctacatctccgaaagcaagctcttctagaagttgatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 20 - protein, 472 aa, Homo sapiens; identical to SEQ ID NO: 18

SEQ ID NO: 21 - DNA, 2108 bp, Homo sapiens
  CDS (415)..(1833)
ctttcaattcctctggcaacaaaccacacactgggatctgacactgtagagtgctttctcttctctttttttgggggggggagggggtgtggttgcatatttaaactctcacgcatttatgtactgaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 22 - protein, 472 aa, Homo sapiens; identical to SEQ ID NO: 18

SEQ ID NO: 23 - DNA, 1814 bp, Homo sapiens
  CDS (121)..(1539)
aagttgctgagacaagggaagagagatgaggaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 24 - protein, 472 aa, Homo sapiens; identical to SEQ ID NO: 18

SEQ ID NO: 25 - DNA, 1989 bp, Homo sapiens
  CDS (296)..(1714)
atgacattattagttctgccactggtaggcattagaagcaagaaaagggagacggaccgaggaagccactttggtgaaacaaaaagaaaagcatttgtttatttagaacgggcaaaatgatacgtttcagtgggtgttttctttgtactttgatctttttgtactgatatttaagcttctgttttatgatctctttctaatgatagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 26 - protein, 472 aa, Homo sapiens; identical to SEQ ID NO: 18

SEQ ID NO: 27 - DNA, 2625 bp, Rattus norvegicus
  CDS (234)..(1652)
tggacttgtactctctcctcggatggctagctgattacttctgtgtagtagcttacaaatgattcgaagtgttgaaactttctgagtctcaatgaactatttctcacacaactcagatactgctgttcatgcatgaattagttgaaccaggccacatagaaagcattgtaattgtacctgtgagttggcaagaagcaagtgctcttccttgattctgctgcacgaggaggagaatgggctgcgatcggaactgtgggctcattactggagccgttattggtgctgtcctggctgtgtttggaggcattctcatgccggttggagacctactcattgagaagacaatcaaaagggaagttgtccttgaagaaggaaccattgctttcaaaaactgggtgaaaacgggcaccactgtgtacagacagttttggatctttgacgtgcaaaacccagaggaagtggcaaagaatagcagcaagatcaaggttaaacagagaggtccttacacatacagagttcgttatttagccaaggaaaatataactcaggaccccaaggacagcactgtctcttttgtacaacccaatggagccatctttgagccttcactgtctgttggaacagagaatgacaacttcacagttctcaatctggctgtggcagctgcaccacatatctacacaaactcatttgttcaaggtgtgctcaacagccttatcaaaaagtccaagtcttctatgttccaaacacgaagtttgaaggaactcttgtggggttacaaagatccattcttgagtttggttccatatcctataagtaccacagttggtgtgttttatccttacaataacactgtagatggagtttataaagttttcaatggaaaggataacataagcaaggttgccataattgatacctataaagggaaaaggaatttgtcctattgggaaagttattgcgacatgattaatggcacagatgcagcctcctttccaccttttgttgagaagtctcgaacactgaggttcttttcctctgacatttgcaggtccatctatgctgtgtttggatctgaagtgaaccttaaaggaatccccgtgtacagatttgttcttccagccaacgcctttgcctccccactccagaacccagacaaccactgtttctgcactgaaaaagtaatctcaaataactgtacgtcgtatggtgtgctggacattggcaagtgcaaagaaggaaagcctgtgtacatttctcttccacatttcctacatgcaagtcctgatgtctcagaacctatcgaaggcttgaatcctaacgaagatgagcataggacatacttggatgtggaacccataactggattcactctacagtttgcaaaacgactgcaggtcaacatactggtcaagccagctagaaaaatagaagcactgaagaatctgaagagaccttacattgtacctatactgtggctaaatgagactgggaccatcggcgatgagaaagcagaaatgttcagaaaccaagtgaccgggaaaataaagctcctgggcctggttgagatggtcttacttggtgttggagtagtgatgtttgttgcttttatgatttcatactgtgcttgcagatctaagaatggaaaataagtagtggatgagcctacattatgcactagctacatttttggtaaaaccaatctccaaaacgaagacttaagacatgcttgtttttataaaacacacctatctgtagttgaagaaacggtggtgtgcgcgctctctctcttattgcagatatatatttatttatatattgcaataagccacagcatatttcgaaaagattaatatgtcactaagcctatattttttaataaaatcttgtattttgttaagtccatcatctgcaactgagtggacatcaatttctgcagaactaattatcttttttggttctgatttactgattttttttcctgttggcaaatttcaagaatgtatatattctaagaaacgctttgttcctcatcgaagtaaactgttatcatgtctggggtggccctttcatttatagcaaatgttccttgtgactgtcagcacatgatatgtcataaggattatatcattttaaagatttaaggatgaaaaatgaacaattcacatatgaaccattgttgatatattgtttaatcctctccctctctggtgtccttggcaacaacaaggccaggtatcacagatacttttttctttttactttcttacacagagcttatatgttctgttcctcgccatgaaatgaactatttttagcacattttagctctttattttaagtatgttgtcaagttccatgctgcctagctcttttgaaaactgagtaggtttttctctttctgctcagccgcaactaatgtaacttcagagagctgttatagtgttaaaagatgtaatttataataaatggattatgatatagaatcttaaaaaagctagaattggctttaaatatgtatttgtggtaatatattctgcttttataatcacccagaaataactggtttctaacattaaagatgttcttaaattcaaaaaaaaaaaaaaaaaaaaaaaaaaaaaa

SEQ ID NO: 28 - protein, 472 aa, Rattus norvegicus
MGCDRNCGLITGAVIGAVLAVFGGILMPVGDLLIEKTIKREVVLEEGTIAFKNWVKTGTTVYRQFWIFDVQNPEEVAKNSSKIKVKQRGPYTYRVRYLAKENITQDPKDSTVSFVQPNGAIFEPSLSVGTENDNFTVLNLAVAAAPHIYTNSFVQGVLNSLIKKSKSSMFQTRSLKELLWGYKDPFLSLVPYPISTTVGVFYPYNNTVDGVYKVFNGKDNISKVAIIDTYKGKRNLSYWESYCDMINGTDAASFPPFVEKSRTLRFFSSDICRSIYAVFGSEVNLKGIPVYRFVLPANAFASPLQNPDNHCFCTEKVISNNCTSYGVLDIGKCKEGKPVYISLPHFLHASPDVSEPIEGLNPNEDEHRTYLDVEPITGFTLQFAKRLQVNILVKPARKIEALKNLKRPYIVPILWLNETGTIGDEKAEMFRNQVTGKIKLLGLVEMVLLGVGVVMFVAFMISYCACRSKNGK

(19) United States
(12) Patent Application Publication
(10) Pub. No.: US 2010/0279941 A1
(43) Pub. Date: Nov. 4, 2010

(54) CD36 MODULATION AND USES THEREOF

(76) Inventors: Huy Ong, Ville Mont-Royal (CA); Sylvie Marleau, Rosemere (CA); André Carpentier, Sherbrooke (CA); Valérie L. Bessi, Terrebonne (CA); Sébastien Ménard, Sherbrooke (CA)

Correspondence Address: GOUDREAU GAGE DUBUC, 2000 MCGILL COLLEGE, SUITE 2200, MONTREAL, QC, H3A 3H3, CA

(21) Appl. No.: 12/771,116

(22) Filed: Apr. 30, 2010

Related U.S. Application Data
(60) Provisional application No. 61/174,671, filed on May 1, 2009.

(30) Foreign Application Priority Data
May 1, 2009 (CA) 2,665,302

(51) Int. Cl. A61K 38/08; A61K 38/07; A61K 38/06; G01N 33/566; C12Q 1/02; A61P 9/10

(52) U.S. Cl. 514/17; 514/18; 435/7.2; 435/29

(57) ABSTRACT

Methods, uses, kits and products are described for the prevention and treatment of ischemia-associated cardiopathies such as myocardial ischemia/reperfusion (I/R) injury, based on the selective modulation of CD36.

### CROSS REFERENCE TO RELATED APPLICATIONS

This application claims the benefit of U.S. provisional application serial No. 61/174,671 and claims priority from Canadian application No. 2,665,302, both filed on May 1, 2009, which are incorporated herein by reference in their entirety.

### SEQUENCE LISTING

This application contains a Sequence Listing in computer readable form entitled 12810_{—}314_ST25, created Apr. 27, 2010 having a size of 82 kb, which is incorporated herein by reference.

### TECHNICAL FIELD

The present invention relates to the prevention and treatment of ischemic-related conditions, and more particularly to ischemic-related cardiopathies such as coronary heart disease, myocardial infarction and myocardial ischemia/reperfusion (I/R).

### BACKGROUND ART

Despite advances in the management of ischemic heart disease (IHD), it remains the world's greatest killer, and the escalating emergence of associated risk factors such as obesity and diabetes is likely to influence the incidence of IHD-related morbidity/mortality over the next decades [Poirier et al., 2006; St Pierre et al., 2005]. Myocardial ischemia-reperfusion (I/R) is associated with metabolic and biochemical alterations that may potentiate ventricular tissue damage and dysfunction, such as increased circulating levels of nonesterified free fatty acids (NEFA) during and following heart ischemia [Kurien and Oliver, 1971; Mueller and Ayres, 1978]. One of the regulators of fatty acid uptake in the heart is the fatty acid translocase (FAT)/CD36 protein, following its subcellular relocation from intracellular depots to sarcolemma [Luiken et al., 2003; Bonen et al., 2004; Koonen et al., 2005; Chabowski et al., 2004; Luiken et al., 2002; Luiken et al., 2004; Bastie et al., 2004], in response to stimuli involving activated 5′ AMP-activated protein kinase (AMPK) and Akt (protein kinase B) [Schwenk et al., 2008].

It has been shown that CD36 deficiency does not compromise heart function or energetics in working hearts following ischemia/reperfusion (I/R) ex vivo, as a result of compensatory increases in glucose oxidation rates [Kuang et al., 2004]. Furthermore, recent studies have shown that although CD36 is abundant in cardiac mitochondria, it does not play an essential role in the uptake and oxidation of long chain fatty acids (LCFA), nor the export of LCFA from the matrix [King et al., 2007].

LCFA and their mitochondrial oxidative metabolites are the primary source of energy utilized in normal adult hearts (carbohydrates accounting for most of the remainder), a reduced oxygen supply to the heart is associated with impaired myocardial LCFA uptake and oxidation, with a relative increase in anaerobic glycolysis. During severe ischemia, pyruvate accumulation, which cannot be oxidized and is reduced into lactate, as well as the accumulation of protons (from the splitting of ATP), accounts for intracellular acidosis as a consequence of increases in H⁺/Na⁺ and Na⁺/Ca⁺⁺ exchangers activity. This leads to calcium overload, electrical instability, cardiac and mitochondrial dysfunction [Sambandam and Lopaschuk, 2003].

Reperfusion of ischemic heart, although important to tissue survival, is associated with high rates of LCFA oxidation and potentially more tissue injury. Indeed, in that context LCFA oxidation will predominate over glucose oxidation, owing to the increased LCFA availability (through catecholamine-mediated intracellular adipose tissue lipolysis or lipoprotein lipase-driven intravascular triglyceride lipolysis), and a concomitant decrease in pyruvate dehydrogenase (PDH) activity. The resulting decrease in glucose-derived acetyl CoA creates an imbalance between glucose oxidation and glycolysis end product formation, thereby promoting lactate and proton accumulation (Randle cycle) [Dolinsky and Dyck, 2006; Kudo et al., 1996]. Myocardial I/R also activates the metabolic sensor AMPK, the latter mediating the phosphorylation and inhibition of acetyl-CoA carboxylase (ACC), thereby preventing malonyl-CoA formation and setting free carnitine palmitoyltransferase-1 (CPT-1) to catalyze the transport of LCFA through the mitochondrial membrane.

Up until now, the proposed metabolic approaches to prevent/treat myocardial I/R injury include stimulation of pyruvate dehydrogenase with dicholoroacetate (the benefits of which is limited by a short half-life); the inhibition of adipocyte lipolysis (with beta-blockers, nicotinic acid and derivatives); the inhibition of CPT-1 with perhexilline (outlawed in many countries due to its narrow therapeutic index) or malonyl CoA decarboxylase; and use of LCFA oxidation inhibitor (trimetazidine, ranozaline) and carnitine biosynthesis inhibitor (mildronate) [Wang and Lopaschuk, 2007]. Until now, these approaches have been associated with limited therapeutic success.

Thus, there is a need for novel methods and products for the prevention/treatment of ischemia cardiopathies such as myocardial (I/R).

The present description refers to a number of documents, the content of which is herein incorporated by reference in their entirety.

### SUMMARY OF THE INVENTION

The present invention relates to the modulation of CD36 activity, and uses thereof for the prevention and treatment of ischemia-associated diseases/conditions such as ischemia-associated heart conditions, and more particularly for the prevention and treatment of myocardial I/R injury

In a first aspect, the present invention provides a method for preventing and/or treating an ischemia-related heart condition in a subject comprising administering an effective amount of a selective CD36 ligand to said subject.

In another aspect, the present invention provides a use of a selective CD36 ligand for preventing and/or treating an ischemia-related heart condition in a subject.

In another aspect, the present invention provides a use of a selective CD36 ligand for the preparation of a medicament for preventing and/or treating an ischemia-related heart condition in a subject.

In another aspect, the present invention provides a selective CD36 ligand for preventing and/or treating an ischemia-related heart condition in a subject.

In another aspect, the present invention provides a selective CD36 ligand for use in preventing and/or treating an ischemia-related heart condition in a subject.

In another aspect, the present invention provides a selective CD36 ligand for the preparation of a medicament for preventing and/or treating an ischemia-related heart condition in a subject.

In another aspect, the present invention provides a composition for preventing and/or treating an ischemia-related heart condition in a subject, said composition comprising the above-mentioned selective CD36 ligand and a pharmaceutically acceptable carrier or excipient.

In another aspect, the present invention provides a composition for use in preventing and/or treating an ischemia-related heart condition in a subject, said composition comprising the above-mentioned selective CD36 ligand and a pharmaceutically acceptable carrier or excipient.

In another aspect, the present invention provides a method for determining whether a test compound may be useful for preventing and/or treating an ischemia-related heart condition, said method comprising determining the binding of said compound to a CD36 polypeptide or a fragment thereof, wherein the binding of said compound to said CD36 polypeptide or fragment thereof is indicative that said compound may be useful for preventing and/or treating said ischemia-related heart condition.

In another aspect, the present invention provides a method for determining whether a test compound may be useful for preventing and/or treating an ischemia-related heart condition, said method comprising contacting said test compound with a cell expressing a CD36 polypeptide or a fragment thereof; and measuring a CD36-associated activity, wherein a modulation of said CD36-associated activity in the presence of said test compound is indicative that said test compound may be useful for preventing and/or treating said ischemia-related heart condition.

In an embodiment, the above-mentioned ischemia-related heart condition is myocardial ischemia/reperfusion (I/R).

In another embodiment, the above-mentioned method or use further comprises (a) decreasing plasma nonesterified free fatty acids (NEFA) levels; (b) decreasing infarct size; (c) reducing myocardial NEFA uptake; (d) decreasing myocardial oxidative metabolism; (e) decreasing myocardial blood flow; (f) increasing end-diastolic and end-systolic ventricular volumes; (g) increasing stroke volume; (h) increasing the relative ratio of phosphorylated Akt to total Akt in myocardial cells; (i) increasing the relative ratio of phosphorylated AMPK to total AMPK in myocardial cells; (j) decreasing myocardial leukocyte accumulation; (k) decreasing circulating blood leukocyte activation; or (l) any combination of (a) to (k).

In an embodiment, the above-mentioned selective CD36 ligand is a peptide-like compound.

In a further embodiment, the above-mentioned peptide-like compound is of general Formula I:

R⁸—X—R⁹ (I)

wherein

R⁸ is absent or is a N-terminal modification;

R⁹ is absent or is a C-terminal modification; and

X is a peptide-like domain.

In an embodiment, the above-mentioned X comprises an aza-amino acid such that said peptide-like domain comprises an aza inter-amino acid linkage.

In another embodiment, the above-mentioned X comprises at least one D-amino acid.

In an embodiment, the above-mentioned X is a peptide-like domain of formula II:

| | |
|---|---|
| Xaa¹-Xaa²-Xaa³-Xaa⁴-Xaa⁵-Xaa⁶ (II) | (SEQ ID NO: 1) |

wherein

Xaa¹ is L-His, D-His, Ala, Phe, a hydrocinnamyl group, a [(2S, 5S)-5-amino-1,2,3,4,6,7-hexahydro-azepino (3,2,1-hi)indol-4-one-2-carboxylic acid group (HAIC group), or a 2-R-(2p,5p,8p)-8-amino-7-oxo-4-thia-1-aza-bicyclo 3.4.0 nonan-2-carboxylate group (ATAB group);

Xaa² is AzaPhe, AzaTyr, D-Trp or 2MeD-Trp (a D-tryptophan residue methylated at position 2, also referred to as D-Mrp);

Xaa³ is Ala, AzaLeu, AzaPro, AzaGly or D-Lys;

Xaa⁴ is Ala, Trp, AzaTyr or AzaPhe;

Xaa⁵ is D-Phe, Ala or D-Ala; and

Xaa⁶ is Lys or Ala.

In an embodiment, the above-mentioned Xaa⁴ is Trp. In another embodiment, the above-mentioned Xaa⁵ is D-Phe. In yet another embodiment, the above-mentioned Xaa⁶ is Lys.

In another embodiment, the above-mentioned X is:

(SEQ ID NO: 2)
(a) (D/L)His-AzaPhe-Ala-Ala-DPhe-Lys;
(SEQ ID NO: 3)
(b) Ala-AzaPhe-Ala-Trp-DPhe-Lys;
(SEQ ID NO: 4)
(c) His-AzaTyr-Ala-Trp-DPhe-Ala;
(SEQ ID NO: 5)
(d) Ala-AzaTyr-Ala-Trp-DPhe-Lys;
(SEQ ID NO: 6)
(e) His-DTrp-AzaLeu-Trp-Ala-Lys;
(SEQ ID NO: 7)
(f) His-DTrp-AzaLeu-Ala-DPhe-Lys;
(SEQ ID NO: 8)
(g) Phe-DTrp-Ala-AzaTyr-DPhe-Lys;
(SEQ ID NO: 9)
(h) Ala-DTrp-Ala-AzaTyr-DPhe-Lys;
(SEQ ID NO: 10)
(i) Hydrocinnamyl-DTrp-Ala-AzaTyr-DPhe-Lys;
(SEQ ID NO: 11)
(j) Ala-DTrp-azaLeu-Trp-DPhe-Lys;
(SEQ ID NO: 12)
(k) Ala-DTrp-Ala-AzaPhe-DPhe-Lys;
(SEQ ID NO: 13)
(l) His-DTrp-AzaPro-Trp-DPhe-Lys;
(SEQ ID NO: 14)
(m) His-DTrp-AzaGly-Trp-DPhe-Ala;
(SEQ ID NO: 15)
(n) HAIC-2MeDTrp-DLys-Trp-DPhe-Lys;
or
(SEQ ID NO: 16)
(o) ATAB-2MeDTrp-DLys-Trp-DPhe-Lys.

In a further embodiment, the above-mentioned X is Ala-AzaPhe-Ala-Trp-DPhe-Lys (SEQ ID NO:3). In a further embodiment, the above-mentioned X is HAIC-2MeDTrp-DLys-Trp-DPhe-Lys (SEQ ID NO:15). In a further embodiment, the above-mentioned X is Ala-DTrp-Ala-AzaPhe-DPhe-Lys (SEQ ID NO:12). In a further embodiment, the above-mentioned X is His-DTrp-AzaPro-Trp-DPhe-Lys (SEQ ID NO:13).

In an embodiment, the above-mentioned R⁹ is NH₂.

In an embodiment, the above-mentioned CD36 polypeptide or fragment thereof is a human CD36 polypeptide or a fragment thereof.

In another embodiment, the above-mentioned CD36 polypeptide or fragment thereof is expressed at the surface of a cell.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A to 1C show a schematic representation of the experimental protocols performed in mice pre-treated subcutaneously (s.c.) for 14 days with either 0.9% NaCl (vehicle) or 300 μg/kg/d of EP 803717 (HAIC-2MeDTrp-DLys-Trp-D-Phe-Lys-NH₂). FIG. 1A: The mice underwent transient (30 minutes) left coronary artery ligation (LCAL) surgery of the, with a 30-minute left anterior descending (LAD) coronary artery, followed by 6 or 48 hours of reperfusion. FIG. 1B: [¹¹C]-acetate was infused in mice after 5 hours of reperfusion to determine myocardial oxidative rate followed 30 minutes later by an intravenous (i.v.) infusion of [¹⁸F]-fluoro-deoxyglucose (FDG) or [¹⁸F]-fluoro-thia-6-heptadecanoic acid (FTHA) for positron emission tomography (PET) analysis. FIG. 1C: [¹⁴C]-palmitate was infused in mice after 5 hours of reperfusion and were sacrificed at 6 hours;

FIG. 2 shows infarct area (IA) and area at risk (AAR) of the left ventricle (LV) after 30-min LCAL and 48 hours reperfusion. Representative photographs of mid-ventricular myocardium from CD36^{+/+} vehicle-treated mice (panel A), CD36^{+/+} EP 80317-treated mice for 14 days (panel B), CD36^{-/-} vehicle-treated mice (panel C) and CD36^{-/-} EP 80317-treated mice (panel D). Panel E: Bar graphs of AAR to LV ratio (AAR/LV), infarct area to left ventricle ratio (IA/LV) and infarct area to AAR (IA/AAR) in CD36^{+/+} mice treated with 0.9% NaCl (vehicle) (n=5) and CD36^{+/+} EP 80317-treated (n=6) mice. Panel F: Bar graphs of IA/AAR, IA/LV and AAR/LV in CD36^{+/+} mice treated with vehicle (n=6) and CD36^{-/-} mice treated with EP 80317 (n=5). *: p<0.05 compared to 0.9% NaCl-treated mice. Panel G: Bar graphs of AAR to LV ratio (AAR/LV), infarct area to left ventricle ratio (IA/LV) and infarct area to AAR (IA/AAR) in CD36^{+/+} mice treated for 14 days with 0.9% NaCl (vehicle) (n=4) and CD36^{+/+} CP1A(IV)-treated (300 μg/kg/d) (n=5) mice. Data are mean±SEM;

FIG. 3 shows myocardial plasma NEFA fractional uptake (Kᵢ—panel A) and plasma NEFA uptake (Kₘ—panel B) determined by micro-Positron Emission Tomography (μPET) after i.v. injection of [¹⁸F]-fluoro-thia-6-heptadecanoic acid (FTHA) 5.5 hours after coronary artery ligation in CD36^{+/+} (left panel; open bar, 0.9% NaCl, n=7; closed bar, EP 80317, n=6) vs. CD36^{-/-} mice (right panel; open bar, 0.9% NaCl, n=6; closed bar, EP 80317, n=7). Panels C-F: Representative mid-ventricular LabTEP™ transaxial images. ¹, ², ³ and ⁴ indicate p<0.05 for difference vs. bar 1, 2, 3 and 4, respectively, by one-way ANOVA with Newman-Keuls multiple comparison test. Data are expressed as mean±SEM;

FIG. 4 illustrates myocardial metabolic rate of glucose (MMRG) determined by μPET after i.v. injection of [¹⁸F]-fluoro-deoxyglucose (FDG) in CD36^{+/+} (panel A, left section; open bar, 0.9% NaCl, n=7; closed bar, EP 80317, n=5) vs. CD36^{-/-} mice (panel A, right section; open bar, 0.9% NaCl, n=5; closed bar, EP 80317, n=5). Panels B-E: Representative mid-ventricular LabTEP transaxial images. Data are expressed as mean±SEM;

FIG. 5 shows myocardial oxidative metabolism (k₂) determined by μPET after i.v. injection of [¹¹C]-acetate in CD36^{+/+} (left section; open bar, 0.9% NaCl, n=6; closed bar, EP 80317, n=6) vs. CD36^{-/-} mice (right section; open bar, 0.9% NaCl, n=6; closed bar, EP 80317, n=6). ¹, ², ³ and ⁴ indicate P<0.05 for difference vs. bar 1, 2, 3 and 4, respectively, by One-Way ANOVA with Newman-Keuls multiple comparison test. Data are expressed as mean±SEM;

FIGS. 6A to 6D show the estimation of intracardiac ventricular and ejection volumes, and ejection fraction by micro-positron emission tomography PET imaging in mice after transient myocardial ischemia. FIG. 6A: End-diastolic volume; FIG. 6B: End-systolic volume; FIG. 6C: Stroke volume; and FIG. 6D: Ejection fraction; **: p<0.01 compared to 0.9% NaCl-treated WT mice and ##: p<0.01 and ###: p<0.001 compared to EP80317-treated WT mice, by One-Way ANOVA with Newman-Keuls multiple comparison test. Data are expressed as mean±SEM;

FIGS. 7A to 7F show protein and phosphoprotein expression following transient LCAL surgery in CD36^{+/+} and CD36^{-/-} mice. FIG. 7A: phosphorylated and total Akt and AMPK bands in CD36^{+/+} mice (representative of 4-5 mice) and CD36^{-/-} (representative of 5-6 mice) following 6 hours reperfusion. FIG. 7B: phosphorylated and total Akt and AMPK bands in CD36^{+/+} mice (representative of 5 mice) and CD36^{-/-} (representative of 6-8 mice) following 48 hours reperfusion. FIGS. 6C and E: Bar graphs represent the mean values and standard errors of the relative band intensity ratios normalized to the corresponding α-tubulin band intensity at 6 hours post-reperfusion. FIGS. 6D and F: Bar graphs represent the mean values and standard errors of the relative band intensity ratios normalized to the corresponding α-tubulin band intensity at 48 hours post-reperfusion. *: p<0.05, **: p<0.01 compared to 0.9% NaCl-treated mice;

FIGS. 8A to 8D show the effect of a 10-week pretreatment with EP 80317 on leukocyte recruitment and circulating leukocyte activation following transient LCAL and 48 hours reperfusion. FIG. 8A: ventricular leukocyte recruitment in CD36^{+/+} mice (n=5-6). FIG. 8B: ventricular leukocyte recruitment in CD36^{-/-} mice (n=4-5). FIG. 8C: opsonized zymosan-stimulated whole blood chemiluminescence in CD36^{+/+} (n=5-7). FIG. 8D: opsonized zymosan-stimulated whole blood chemiluminescence in CD36^{-/-} mice (n=8-9). *: p<0.05, **: p<0.01 compared to 0.9% NaCl-treated mice;

FIGS. 9A and 9B show the nucleotide (coding sequences shown in bold) sequence of human CD36, transcript variant 1 (SEQ ID NO:17);

FIG. 9C shows the nucleotide (coding sequences shown in bold) sequence of human CD36, transcript variant 2 (SEQ ID NO:19);

FIG. 9D shows the nucleotide (coding sequences shown in bold) sequence of human CD36, transcript variant 3 (SEQ ID NO:21);

FIG. 9E shows the nucleotide (coding sequences shown in bold) sequence of human CD36, transcript variant 4 (SEQ ID NO:23);

FIG. 9F shows the nucleotide (coding sequences shown in bold) sequence of human CD36, transcript variant 5 (SEQ ID NO:25);

FIG. 9G shows the amino acid sequence of human CD36 polypeptide (SEQ ID NO:20);

FIG. 10A shows the nucleotide (coding sequence shown in bold) sequence of rat CD36 (GenBank Accession No. NM_{—}031561; SEQ ID NO: 27);

FIG. 10B shows the amino acid sequence of rat CD36 polypeptide (SEQ ID NO:28);

FIG. 11 shows the binding affinity of azapeptides for CD36 and GHS-R1a;

FIGS. 12A to 12D show representative photographs of TTC-stained mid-left ventricular myocardium from CD36^{+/+} mice treated with vehicle (FIG. 12A), EP 80317 (FIG. 12B), CP-AP-4 (FIG. 12C) and CP-3(iv) (FIG. 12D).

FIG. 12E shows bar graphs depicting infarct area (IA) and area at risk (AAR) of the left ventricle (LV), after 30-min ligation and 48 hours reperfusion. ***, P<0.001 compared to 0.9% NaCl-treated mice;

FIG. 13A shows troponin I (cTnI) levels in plasma of mice at 48 hours post-ischemia that were treated for 2 weeks with 289 nmol/kg of EP 80317 (n=6), CP-AP-4 (n=8) and CP-3(iv) (n=12). **, P<0.01; ***, P<0.001 compared to 0.9% NaCl-treated mice;

FIG. 13B shows a correlation between plasma levels of cTnI and infarct area;

FIG. 14 shows a correlation between ROS generation in LV homogenate and whole blood at 6 hours post-ischemia, in mice treated for 2 weeks with either vehicle or EP 80317, CP-AP-4 and CP-3(iv). Peak amplitude values were determined in mm and data were normalized for protein levels (heart homogenates) and circulating leukocyte blood counts (heart and blood chemiluminescence);

FIG. 15 shows lactate levels in mice treated for 2 weeks with either vehicle or EP 80317, CP-AP-4 and CP-3(iv). Blood (5 μl) was withdrawn 5 min following the 6-hour reperfusion period for lactate level determination in mice treated for 2 weeks with 289 nmol/kg of EP 80317, CP-AP-4 and CP-3(iv) (n=4-6 mice per group). *, P<0.05 compared to 0.9% NaCl-treated C57BL/6 mice; and

FIG. 16 shows the effect of a 2-week treatment with 289 nmol/kg of EP 80317, CP-AP-4 and CP-3(iv) on systemic hemodynamics at 6 hours following transient ischemia in C57BL/6 mice (n=5-8 mice per group)

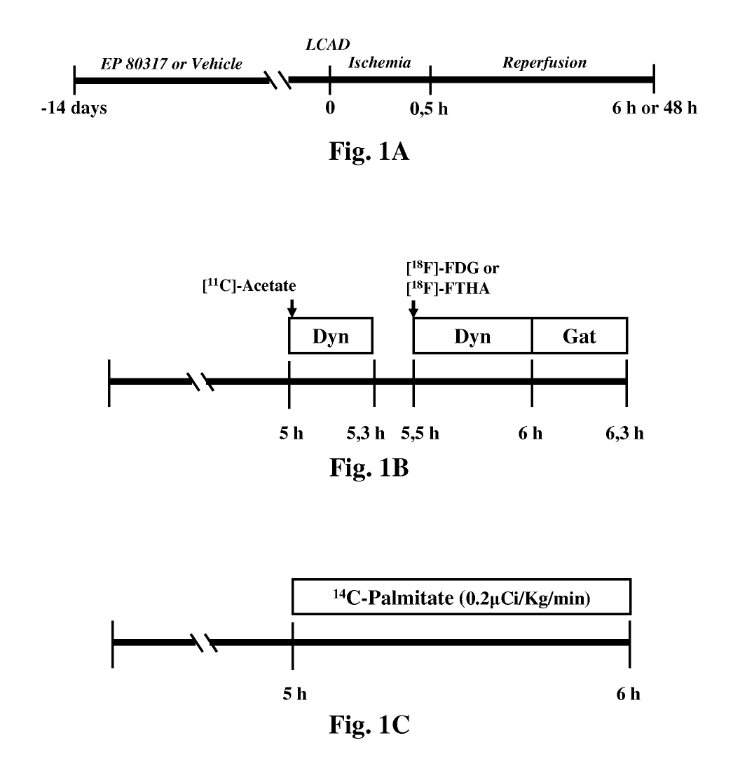

### DISCLOSURE OF INVENTION

Described herein are methods, uses, kits and products for the prevention and treatment of ischemia-related diseases/conditions, and more particularly to ischemia-related heart condition such as myocardial ischemia/reperfusion (I/R), based on changes in/modulation of CD36.

CD36, also known as FAT, SCARB3, GP88, glycoprotein IV (gpIV) and glycoprotein IIIb (gpIIIb), is an integral membrane protein found on the surface of many cell types in vertebrate animals. CD36 is a member of the class B scavenger receptor family of cell surface proteins. CD36 has been shown to bind many ligands including collagen, thrombospondin, erythrocytes parasitized with Plasmodium falciparum, oxidized low density lipoproteins, native lipoproteins, oxidized phospholipids, and long-chain fatty acids.

In the studies described herein, it is shown that administration of selective CD36 ligands shows cardioprotective effect in a mouse model of ischemia/reperfusion. It is demonstrated herein that administration of these ligands is associated with (a) a decrease in plasma nonesterified free fatty acids (NEFA) levels; (b) a decrease in infarct size; (c) a reduction in myocardial NEFA uptake; (d) a decrease in myocardial oxidative metabolism; (e) a decrease in myocardial blood flow; (f) an increase in end-diastolic and end-systolic ventricular volumes; (g) an increase in stroke volume; (h) an increase in the relative ratio of phosphorylated Akt to total Akt in myocardial cells; (i) a transient increase in the relative ratio of phosphorylated AMPK to total AMPK in myocardial cells; (j) a decrease in myocardial leukocyte accumulation; (k) a decrease in circulating blood leukocyte activation; (l) a reduction in cardiac troponin I (cTnI) levels in plasma; and/or (m) a reduction in lactate concentration in blood.

Accordingly, in a first aspect, the present invention provides a method for preventing and/or treating an ischemia-related heart condition in a subject in need thereof comprising administering an effective amount of a selective CD36 ligand to said subject in need thereof.

In another aspect, the present invention provides a method comprising: selecting a subject in need for a prevention and/or treatment of an ischemia-related heart condition; and administering an effective amount of a selective CD36 ligand to said subject in need for a prevention and/or treatment of an ischemia-related heart condition.

In another aspect, the present invention provides a use of a selective CD36 ligand for preventing and/or treating an ischemia-related heart condition in a subject.

In another aspect, the present invention provides a use of a selective CD36 ligand for the preparation of a medicament for preventing and/or treating an ischemia-related heart condition in a subject.

In another aspect, the present invention provides a selective CD36 ligand for preventing and/or treating an ischemia-related heart condition in a subject.

In another aspect, the present invention provides a selective CD36 ligand for the preparation of a medicament for preventing and/or treating an ischemia-related heart condition in a subject.

As used herein, the term “ischemia-related heart condition” (or “ischemic heart disease” or “ischemic cardiomyopathy”) generally refers to any damage and/or dysfunction of the heart (e.g., heart tissue damage and/or dysfunction) associated with ischemia and/or reperfusion, such as myocardial ischemia/reperfusion (I/R), acute myocardial infarction (AMI) and transplantation. For example, ischemia and/or reperfusion are associated with metabolic and biochemical alterations, such as increased circulating levels of nonesterified free fatty acids (NEFA), which in turn causes ventricular tissue damage and dysfunction. In an embodiment, the above-mentioned ischemia-related heart condition is myocardial ischemia/reperfusion (I/R).

As used herein the term “selective CD36 ligand” refers to a molecule which binds specifically to CD36, i.e., exhibits preferential binding to the CD36 receptor relative to another receptor. In an embodiment, the selective CD36 ligand has no or substantially no binding affinity to a ghrelin receptor such as GHS-R1a. “No binding affinity” as used herein refers to a binding affinity corresponding to an IC₅₀ value of about 1×10⁻⁸ M or greater, to a ghrelin receptor such as GHS-R1a. In an embodiment, the selective CD36 ligand induces an intracellular CD36-associated signaling cascade within target cells such as a myocardial cells. In an embodiment, the above-mentioned signal is associated with an increase in the phosphorylation of the serine/threonine protein kinase Akt/PKB (Akt) (e.g., an increase in the ratio of phosphorylated Akt to total Akt) and/or a transient increase in the phosphorylation of AMP-activated protein kinase (AMPK) (e.g., an increase in the ratio of phosphorylated AMPK to total AMPK).

In an embodiment, the above-mentioned selective CD36 ligand has no or substantially no somatotrophic activity (e.g., has no or substantially no growth hormone releasing activity). In an embodiment, the above-mentioned selective CD36 ligand lacks binding activity to, or has low affinity for (e.g., has an IC₅₀ value of about 1×10⁻⁸ M or less), a ghrelin receptor such as GHS-R1a.

In an embodiment, the above-mentioned selective CD36 ligand is a peptide-like compound. As used herein, the term “peptide-like compound” refers to a compound comprising at least two amino acids. In an embodiment, the peptide-like compound comprises amino acids linked by peptide bonds (i.e., an amide bond) such that the backbone of the peptide-like compound has a typical repeating-amine-αCR¹⁰-carbonyl-peptide backbone structure (the αC being the point of attachment for the amino acid side chain R¹⁰). In a further embodiment, the peptide-like compound may comprise one or more aza-amino acids (which results in the αC being replaced by N) such that the compound may comprise within its backbone structure one or more -amine-NR¹⁰-carbonyl-units, wherein R¹⁰ represents the side-chain moiety of the aza-amino acid. In embodiments, the peptide-like compound comprises any combination of amino acids and aza-amino acids.

In a further embodiment, the above-mentioned peptide-like compound is of general. Formula I:

R⁸—X—R⁹ (I)

Wherein

R⁸ is absent or is an N-terminal modification;

R⁹ is absent or is a C-terminal modification; and

X is a peptide-like domain.

As used herein, the term “peptide-like domain” refers to a domain comprising at least two amino acids. In an embodiment, the peptide-like domain comprises amino acids linked by peptide bonds (i.e., an amide bond) such that the backbone of the peptide-like domain has a typical repeating [-amine-αCR^{w}-carbonyl-] peptide backbone structure (the αC being the point of attachment for the amino acid side chain R¹⁰). In a further embodiment, the peptide-like domain may comprise one or more aza-amino acids (which results in the αC being replaced by N) such that the domain may comprise within its backbone structure one or more [-amine-NR¹⁰-carbonyl] units, wherein R¹⁰ represents the side-chain moiety of the aza-amino acid. In embodiments, the peptide-like domain comprises any combination of amino acids and aza-amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, modified amino acids) used in peptide chemistry to prepare synthetic analogs of peptides. Examples of naturally-occurring amino acids are glycine, alanine, valine, leucine, isoleucine, serine, threonine, etc. Other amino acids include for example norleucine, norvaline, cyclohexyl alanine, biphenyl alanine, homophenyl alanine, naphthyl alanine, pyridyl alanine, phenyl alanines substituted at the ortho, para and meta positions with alkoxy, halogen or nitro groups etc. These amino acids are well known in the art of biochemistry/peptide chemistry. In an embodiment, the above-mentioned peptide-like domain (X) comprises at least one D-amino-acid.

Synthetic amino acids providing similar side chain functionality can also be introduced into the peptide. For example, aromatic amino acids may be replaced with D- or L-naphthylalanine, D- or L-phenylglycine, D- or L-2-thienylalanine, D- or L-1-, 2-, 3-, or 4-pyrenylalanine, D- or L-3-thienylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine D-or L-p-methoxybiphenylalanine, D- or L-2-indole(alkyl)alanines, and D- or L-alkylalanines wherein the alkyl group is substituted or unsubstituted methyl, ethyl, propyl, hexyl, butyl, pentyl, isopropyl, iso-butyl, or iso-pentyl.

Non-carboxylate amino acids can be made to possess a negative charge, as provided by phosphono- or sulfated (e.g., —SO₃H) amino acids, which are to be considered as non-limiting examples.

Other substitutions may include unnatural alkylated amino acids, made by combining an alkyl group with any natural amino acid. Basic natural amino acids such as lysine and arginine may be substituted with alkyl groups at the amine (NH₂) functionality. Yet other substitutions include nitrile derivatives (e.g., containing a CN-moiety in place of the CONH₂ functionality) of asparagine or glutamine, and sulfoxide derivative of methionine. In addition, any amide linkage in the peptide may be replaced by a ketomethylene, hydroxyethyl, ethyl/reduced amide, thioamide or reversed amide moieties, (e.g., (—C═O)—CH₂—), (—CHOH)—CH₂—), (CH₂—CH₂—), (—C═S)—NH—), or (—NH—(—C═O) for (—C═O)—NH—)).

Covalent modifications of the above-mentioned peptide-like compound are thus included within the scope of the present invention. Such modifications may be introduced into the above-mentioned peptide-like compound for example by reacting targeted amino acid residues of the polypeptide with an organic derivatizing agent that is capable of reacting with selected side chains or terminal residues. The following examples of chemical derivatives are provided by way of illustration and not by way of limitation.

Cysteinyl residues may be reacted with alpha-haloacetates (and corresponding amines), such as 2-chloroacetic acid or chloroacetamide, to give carboxymethyl or carboxyamidomethyl derivatives. Histidyl residues may be derivatized by reaction with compounds such as diethylprocarbonate e.g., at pH 5.5-7.0 because this agent is relatively specific for the histidyl side chain, and para-bromophenacyl bromide may also be used; e.g., where the reaction is preferably performed in 0.1M sodium cacodylate at pH 6.0. Lysinyl and amino terminal residues may be reacted with compounds such as succinic or other carboxylic acid anhydrides. Other suitable reagents for derivatizing alpha-amino-containing residues include compounds such as imidoesters, e.g. methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with one or several conventional reagents, among them phenylglyoxal, 2,3-butanedione, 1,2-cyclohexanedione, and ninhydrin according to known method steps. Derivatization of arginine residues is typically performed in alkaline conditions because of the high pKa of the guanidine functional group. Furthermore, these reagents may react with the groups of lysine as well as the arginine epsilon-amino group. The specific modification of tyrosinyl residues per se is well-known, such as for introducing spectral labels into tyrosinyl residues by reaction with aromatic diazonium compounds or tetranitromethane. N-acetylimidazol and tetranitromethane may be used to form O-acetyl tyrosinyl species and 3-nitro derivatives, respectively. Tryptophan residues may be methylated at position 2 (sometimes referred to as 2Me-Trp or Mrp).

Carboxyl side groups (aspartyl or glutamyl) may be selectively modified by reaction with carbodiimides (R′—N═C═N—R′) such as 1-cyclohexyl-3-(2-morpholinyl-(4-ethyl)carbodiimide or 1-ethyl-3-(4-azonia-4,4-dimethylpentyl) carbodiimide. Furthermore aspartyl and glutamyl residues may be converted to asparaginyl and glutaminyl residues by reaction with ammonium ions. Glutaminyl and asparaginyl residues may be frequently deamidated to the corresponding glutamyl and aspartyl residues. Other modifications of the above-mentioned peptide analog/azapeptide may include hydroxylation of proline and lysine, phosphorylation of hydroxyl groups of seryl or threonyl residues, methylation of the alpha-amino groups of lysine, arginine, and histidine side chains acetylation of the N-terminal amine, methylation of main chain amide residues (or substitution with N-methyl amino acids) and, in some instances, amidation of the C-terminal carboxyl groups, according to known method steps.

Covalent attachment of fatty acids (e.g., C₆-C₁₈) to the peptide-like compound may confer additional biological properties such as protease resistance, plasma protein binding, increased plasma half-life, intracellular penetration, etc.

In embodiments, the N- and/or C-termini of the above-mentioned peptide-like compound may be modified by addition of (R⁸ and/or R⁹) one or more amino acid(s), amidation, acetylation, acylation or other modifications (e.g., alkylation, alkenylation, alkynylation, arylation, etc.) known in the art. In an embodiment, the amino terminal residue (i.e., the free amino group at the N-terminal end) of the above-mentioned peptide domain is modified (e.g., for protection against degradation). In an embodiment, the modification is acylation with a C₂-C₁₆ acyl group, in a further embodiment, the modification is acetylation.

In an embodiment, the carboxy terminal residue (i.e., the free carboxy group at the C-terminal end) of the above-mentioned peptide-like domain is modified (e.g., for protection against degradation). In an embodiment, the modification is an amidation (i.e., R⁹ is NH₂).

In an embodiment, the above-mentioned peptide-like compound or peptide-like domain contains about 100 amino acids or less. In a further embodiment, the above-mentioned peptide-like compound or peptide-like domain contains about 90 amino acids or less. In a further embodiment, the above-mentioned peptide-like compound or peptide-like domain contains about 80 amino acids or less. In a further embodiment, the above-mentioned peptide-like compound or peptide-like domain contains about 70 amino acids or less. In a further embodiment, the above-mentioned peptide-like compound or peptide-like domain contains about 60 amino acids or less. In a further embodiment, the above-mentioned peptide-like compound or peptide-like domain contains about 50 amino acids or less. In a further embodiment, the above-mentioned peptide-like compound or peptide-like domain contains about 40 amino acids or less. In a further embodiment, the above-mentioned peptide-like compound or peptide-like domain contains about 30 amino acids or less. In a further embodiment, the above-mentioned peptide-like compound or peptide-like domain contains about 20 amino acids or less. In a further embodiment, the above-mentioned peptide-like compound or peptide-like domain contains about 15 amino acids or less. In a further embodiment, the above-mentioned peptide-like compound or peptide-like domain contains about 10 amino acids or less. In a further embodiment, the above-mentioned peptide-like compound or peptide-like domain contains about 9 amino acids or less. In a further embodiment, the above-mentioned peptide-like compound or peptide-like domain contains about 8 amino acids or less. In a further embodiment, the above-mentioned peptide-like compound or peptide-like domain contains about 7 amino acids or less. In a further embodiment, the above-mentioned peptide-like compound or peptide-like domain contains about 6 or more amino acids. In a further embodiment, the above-mentioned peptide-like compound or peptide-like domain contains about 5 or more amino acids.

Peptides and peptide-like compounds can be readily synthesized by automated solid phase procedures well known in the art. Suitable syntheses can be performed by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the peptides may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the peptides are described in Nakagawa et al., J. Am. Chem. Soc. 107: 7087-7092, 1985. Commercial providers of peptide synthesis services may also be used to prepare synthetic peptides in the D- or L-configuration. Such providers include, for example, Advanced ChemTech (Louisville, Ky.), Applied Biosystems (Foster City, Calif.), Anaspec (San Jose, Calif.), and Cell Essentials (Boston, Mass.).

Peptides and peptide-like compounds comprising naturally occurring amino acids encoded by the genetic code may also be prepared using recombinant DNA technology using standard methods. Peptides produced by recombinant technology may be modified (e.g., N-terminal acylation [e.g., acetylation], C-terminal amidation, cyclization/formation of a loop within the peptide [e.g., via formation of a disulphide bridge between Cys residues]) using methods well known in the art. Therefore, in embodiments, in cases where a peptide-like compound described herein contains naturally occurring amino acids encoded by the genetic code, the peptide-like compound may be produced using recombinant methods, and may in embodiments be subjected to for example the just-noted modifications (e.g., acylation, amidation, cyclization). Accordingly, in another aspect, the invention further provides a nucleic acid encoding the above-mentioned peptide-like compound. The invention also provides a recombinant nucleic acid comprising the above-mentioned nucleic acid. The invention also provides a vector comprising the above-mentioned nucleic acid. In yet another aspect, the present invention provides a cell (e.g., a host cell) comprising the above-mentioned nucleic acid and/or vector. The invention further provides a recombinant expression system, vectors and host cells, such as those described above, for the expression/production of the above-mentioned peptide-like compound, using for example culture media, production, isolation and purification methods well known in the art.

Such vectors comprise a nucleic acid sequence capable of encoding such a peptide operably linked to one or more transcriptional regulatory sequence(s). In an embodiment, the peptide is a fusion peptide containing a domain which for example facilitates its purification and/or detection (e.g., His-tag, GST-tag). Nucleic acids may be introduced into cells for expression using standard recombinant techniques for stable or transient expression. Nucleic acid molecules of the invention may include any chain of two or more nucleotides including naturally occurring or non-naturally occurring nucleotides or nucleotide analogues.

“Recombinant expression” refers to the production of a peptide or polypeptide by recombinant techniques, wherein generally, a nucleic acid encoding peptide or polypeptide is inserted into a suitable expression vector which is in turn used to transform/transfect a host cell to produce the protein. The term “recombinant” when made in reference to a protein or a polypeptide refers to a peptide, polypeptide or protein molecule which is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e., by human intervention. Recombinant nucleic acid constructs may for example be introduced into a host cell by transformation/transfection. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The term “vector” refers to a nucleic acid molecule which may be used as a vehicle for transfer of another nucleic acid (e.g., a foreign or heterologous nucleic acid) into a cell. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”.

A recombinant expression vector of the present invention can be constructed by standard techniques known to one of ordinary skill in the art and found, for example, in Sambrook et al. (1989) in Molecular Cloning: A Laboratory Manual. A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation and selection in bacteria and host cells. In addition, the vectors of the present invention may comprise a sequence of nucleotides for one or more restriction endonuclease sites. Coding sequences such as for selectable markers and reporter genes are well known to persons skilled in the art.

A recombinant expression vector comprising a nucleic acid sequence encoding a peptide/polypeptide may be introduced into a host cell, which may include a living cell capable of expressing the protein coding region from the defined recombinant expression vector. The living cell may include both a cultured cell and a cell within a living organism. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (Molecular Cloning: A Laboratory Manual, 2^{nd} Edition, Cold Spring Harbor Laboratory press (1989)), and other laboratory manuals. Methods for introducing DNA into mammalian cells in vivo are also known, and may be used to deliver the vector DNA of the invention to a subject for gene therapy.

“Transcriptional regulatory sequence/element” is a generic term that refers to DNA sequences, such as initiation and termination signals, enhancers, and promoters, splicing signals, polyadenylation signals which induce or control transcription of protein coding sequences with which they are operably linked. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous.

As used herein, the term “transfection” or “transformation” generally refers to the introduction of a nucleic acid, e.g., via an expression vector, into a recipient cell by nucleic acid-mediated gene transfer.

A cell (e.g., a host cell or indicator cell), tissue, organ, or organism into which has been introduced a foreign nucleic acid (e.g., exogenous or heterologous DNA [e.g. a DNA construct]), is considered “transformed”, “transfected”, or “transgenic”. A transgenic or transformed cell or organism also includes progeny of the cell or organism and progeny produced from a breeding program employing a transgenic organism as a parent and exhibiting an altered phenotype resulting from the presence of a recombinant nucleic acid construct. A transgenic organism is therefore an organism that has been transformed with a heterologous nucleic acid, or the progeny of such an organism that includes the transgene. The introduced DNA may be integrated into chromosomal DNA of the cell's genome, or alternatively may be maintained episomally (e.g., on a plasmid). Methods of transfection are well known in the art (see for example, Sambrook et al., 1989, supra; Ausubel et al., 1994 supra).

For stable transfection of mammalian cells, it is known that, depending upon the expression vector and transfection technique used, only a small fraction of cells may integrate the foreign DNA into their genome. In order to identify and select these integrants, a gene that encodes a selectable marker (such as resistance to antibiotics) may be introduced into the host cells along with the gene of interest. As used herein, the term “selectable marker” is used broadly to refer to markers which confer an identifiable trait to the indicator cell. Non-limiting example of selectable markers include markers affecting viability, metabolism, proliferation, morphology and the like. Preferred selectable markers include those that confer resistance to drugs, such as G418, hygromycin and methotrexate. Nucleic acids encoding a selectable marker may be introduced into a host cell on the same vector as that encoding the peptide compound or may be introduced on a separate vector. Cells stably transfected with the introduced nucleic acid may be identified by drug selection (cells that have incorporated the selectable marker gene will survive, while the other cells die).

The peptide-like compound of the invention can be purified by many techniques well known in the art, such as reverse phase chromatography, high performance liquid chromatography (HPLC), ion exchange chromatography, size exclusion chromatography, affinity chromatography, gel electrophoresis, and the like. The actual conditions used to purify a particular peptide or peptide analog will depend, in part, on synthesis strategy and on factors such as net charge, hydrophobicity, hydrophilicity, and the like, and will be apparent to those of ordinary skill in the art. For affinity chromatography purification, any antibody which specifically binds the peptide-like compound may for example be used.

In an embodiment, the above-mentioned peptide-like domain (X) comprises an aza-amino acid such that said peptide domain comprises an aza inter-amino acid linkage. Such azapeptide compounds as well as methods for producing same, are described, for example, in PCT publication No. WO 08/154738. For example, azapeptide compounds may synthesized according to well known methods using Fmoc-protected aza-amino acid chlorides to acylate the peptide chain. Removal of the Fmoc group and subsequent coupling of the next amino acid, typically by way of the Fmoc-amino acid chloride, embedded selectively the aza-amino acid residue within the peptide chain.

In an embodiment, the above-mentioned selective CD36 ligand is an azapeptide compound of Formula V:

A-(Xaa)ₐ-N(R^{A})—N(R^{B})—C(O)-(Xaa′)_{b}-B (V)

Wherein

a is an integer from 0 to 5;

b is an integer from 0 to 5;

Xaa and Xaa′ are each any D- or L-amino acid residue, or an aza-amino acid residue;

when a or b is 2 or more, the Xaa or Xaa′ moieties may independently comprise two or more residues therein, whereby each residue may independently be a D- or L-amino acid residue, or an aza-amino acid residue;

A is H, a C₁-C₆ alkyl, a C₂-C₆ alkenyl, a C₂-C₄ alkynyl, a C₃-C₇ cycloalkyl, a haloalkyl, a heteroalkyl, an aryl, a heteroaryl, a heteroalkyl, a heterocyclyl, a heterobicyclyl, C(O)R³, SO₂R³, C(O)OR³, or C(O)NR⁴R⁵, wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;

B is OH, OR³, or NR⁴R⁵;

R^{A} and R^{B} are independently chosen from H, C₁-C₆ alkyl, C₂-C₆ alkenyl, C₂-C₆ alkynyl, C₃-C₇ cycloalkyl, C₅-C₇ cycloalkenyl, haloalkyl, heteroalkyl, aryl, heteroaryl, heterobicyclyl, heterocyclyl, or an amino acid side chain, wherein the alkyl, alkenyl, alkynyl and the cycloalkyl and cycloalkenyl are optionally substituted with one or more R¹ substituents; and wherein the aryl, the heteroaryl, the heterocyclyl and the heterobicyclyl are optionally substituted with one or more R² substituents, or alternatively, R^{A} and R^{B} together with the nitrogen to which each is bonded form a heterocyclic or a heterobicyclic ring;

R¹ is a halogen, NO₂, CN, a haloalkyl, a C₃-C₇ cycloalkyl, an aryl, a heteroaryl, a heterocyclyl, a heterobicyclyl, OR⁶, S(O)₂R³, NR⁴R⁵, NR⁴S(O)₂R³, COR⁶, C(O)OR⁶, CONR⁴R⁵, S(O)₂NR⁴R⁵, OC(O)R⁶, SC(O)R³, NR⁶C(O)NR⁴R⁵, a heteroalkyl, NR⁶C(NR⁶)NR⁴R⁵, or C(NR⁶)NR⁴R⁵; wherein the the aryl, heteroaryl, heterocyclyl, and heterobicyclyl are optionally substituted with one or more R² substituents;

R² is a halogen, NO₂, CN, a C₁-C₆ alkyl, a C₂-C₆ alkenyl, a C₂-C₄ alkynyl, a C₃-C₇ cycloalkyl, a haloalkyl, OR⁶, NR⁴R⁵, SR⁶, COR⁶, C(O)OR⁶, S(O)₂R³, CONR⁴R⁵, S(O)₂NR⁴R⁵, an aryl, a heteroaryl, a heterocyclyl, a heterobicyclyl, a heteroalkyl, NR⁶C(NR⁶)NR⁴R⁵, or C(NR⁶)NR⁴R⁵, wherein the aryl, the heteroaryl, the heterocyclyl, and the heterobicyclyl are optionally substituted with one or more R⁷ substituents;

R³ is a C₁-C₆ alkyl, a C₂-C₆ alkenyl, a C₂-C₄ alkynyl, a C₃-C₇ cycloalkyl, a haloalkyl, an aryl, a heteraryl, a heterocyclyl, or a heterobicyclyl, wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;

R⁴ and R⁵ are independently chosen from H, a C₁-C₆ alkyl, a C₂-C₆ alkenyl, a C₂-C₆ alkynyl, an aryl, a heteroaryl, or a heterocyclyl, or R⁴ and R⁵ together with the nitrogen to which they are bonded form a heterocyclic ring;

R⁶ is H, a C₁-C₆ alkyl, a C₂-C₆ alkenyl, a C₂-C₆ alkynyl, an aryl, a heteroaryl, or a heterocyclyl;

R⁷ is a halogen, NO₂, CN, a C₁-C₆ alkyl, a C₂-C₆ alkenyl, a C₂-C₄ alkynyl, a C₃-C₇ cycloalkyl, a haloalkyl, OR⁶, NR⁴R⁵, SR⁶, COR⁶, C(O)OR⁶, S(O)₂R³, CONR⁴R⁵, S(O)₂NR⁴R⁵, heteroalkyl, NR⁶C(NR⁶)NR⁴R⁵, or C(NR⁶)NR⁴R⁵;

or a salt thereof, or a prodrug thereof.

In an embodiment, the above-mentioned X is a peptide-like domain of formula II:

| | |
|---|---|
| Xaa¹-Xaa²-Xaa³-Xaa⁴-Xaa⁵-Xaa⁶ (II) | (SEQ ID NO: 1) |

wherein

Xaa¹ is L-His, D-His, Ala, Phe, a hydrocinnamyl group, a [(2S,5S)-5-amino-1,2,3,4,6,7-hexahydro-azepino (3,2,1-hi)indol-4-one-2-carboxylic acid group (HAIC group), or a 2-R-(2p,5p,8p)-8-amino-7-oxo-4-thia-1-aza-bicyclo 3.4.0 nonan-2-carboxylate group (ATAB group);

Xaa² is AzaPhe, AzaTyr, D-Trp or 2MeD-Trp (a D-tryptophan residue methylated at position 2, also referred to as D-Mrp);

Xaa³ is Ala, AzaLeu, AzaPro, AzaGly or D-Lys;

Xaa⁴ is Ala, Trp, AzaTyr or AzaPhe;

Xaa⁵ is D-Phe, Ala or D-Ala; and

Xaa⁶ is Lys or Ala.

In an embodiment, the above-mentioned Xaa⁴ is Trp. In another embodiment, the above-mentioned Xaa⁵ is D-Phe. In yet another embodiment, the above-mentioned Xaa⁶ is Lys.

In another embodiment, the above-mentioned X is: (a) (D/L)His-AzaPhe-Ala-Ala-DPhe-Lys (SEQ ID NO:2); (b) Ala-AzaPhe-Ala-Trp-DPhe-Lys (SEQ ID NO:3); (c) His-AzaTyr-Ala-Trp-DPhe-Ala (SEQ ID NO:4); (d) Ala-AzaTyr-Ala-Trp-DPhe-Lys (SEQ ID NO:5); (e) His-DTrp-AzaLeu-Trp-Ala-Lys (SEQ ID NO:6); (f) His-DTrp-AzaLeu-Ala-DPhe-Lys (SEQ ID NO:7); (g) Phe-DTrp-Ala-AzaTyr-DPhe-Lys (SEQ ID NO:8); (h) Ala-DTrp-Ala-AzaTyr-DPhe-Lys (SEQ ID NO:9); (i) Hydrocinnamyl-DTrp-Ala-AzaTyr-DPhe-Lys (SEQ ID NO:10); (j) Ala-DTrp-azaLeu-Trp-DPhe-Lys (SEQ ID NO:11); (k) Ala-DTrp-Ala-AzaPhe-DPhe-Lys (SEQ ID NO:12); (I) His-DTrp-AzaPro-Trp-DPhe-Lys (SEQ ID NO:13); (m) His-DTrp-AzaGly-Trp-DPhe-Ala (SEQ ID NO:14); (n) HAIC-2MeDTrp-DLys-Trp-DPhe-Lys (SEQ ID NO:15); or (o) ATAB-2MeDTrp-DLys-Trp-DPhe-Lys (SEQ ID NO:16).

In a further embodiment, the above-mentioned peptide-like compound is:

(a) (D/L)His-AzaPhe-Ala-Ala-DPhe-Lys-NH₂

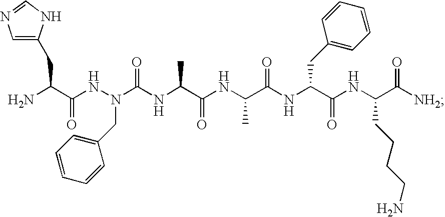

(b) Ala-AzaPhe-Ala-Trp-DPhe-Lys-NH₂

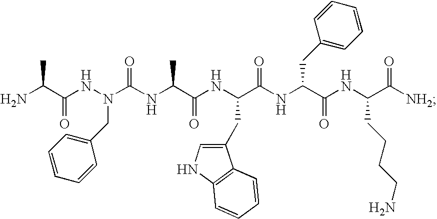

(c) His-AzaTyr-Ala-Trp-DPhe-Ala-NH₂

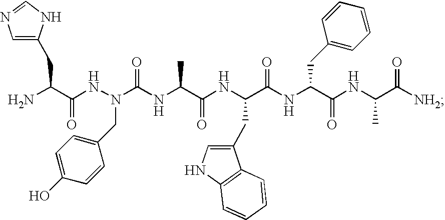

(d) Ala-AzaTyr-Ala-Trp-DPhe-Lys-NH₂

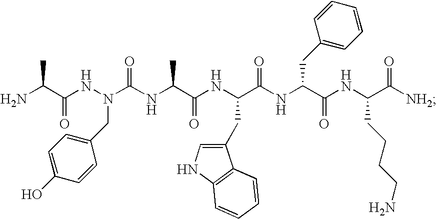

(e) His-DTrp-AzaLeu-Trp-Ala-Lys-NH₂

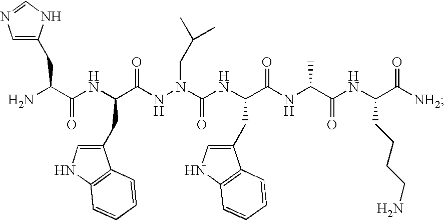

(f) His-DTrp-AzaLeu-Ala-DPhe-Lys-NH₂

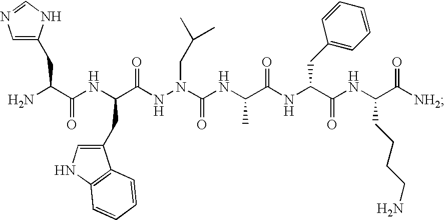

(g) Phe-DTrp-Ala-AzaTyr-DPhe-Lys-NH₂

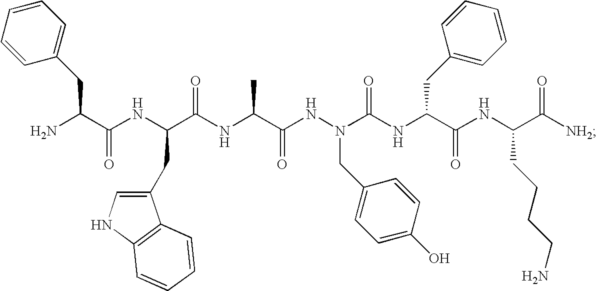

(h) Ala-DTrp-Ala-AzaTyr-DPhe-Lys-NH₂

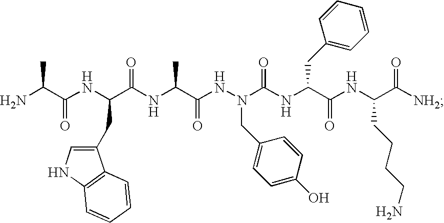

(i) Hydrocinnamyl-DTrp-Ala-AzaTyr-DPhe-Lys-NH₂

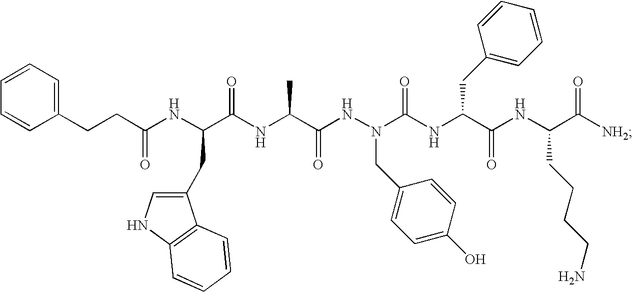

(j) Ala-DTrp-azaLeu-Trp-DPhe-Lys-NH₂

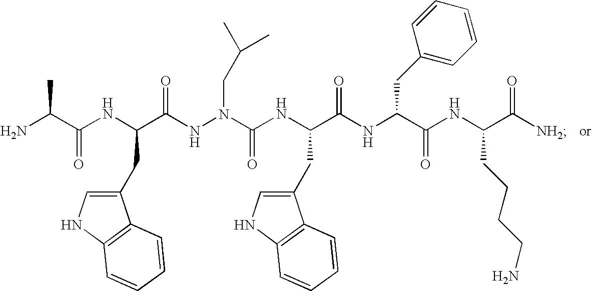

(k) Ala-DTrp-Ala-AzaPhe-DPhe-Lys-NH₂;

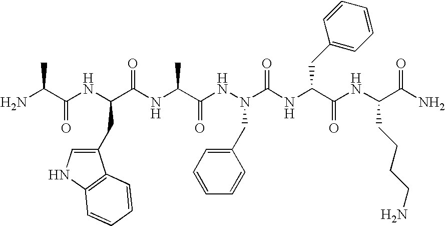

(I) His-DTrp-AzaPro-Trp-DPhe-Lys-NH₂:

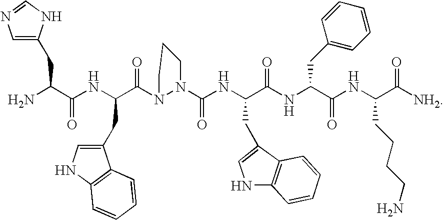

(m) His-DTrp-AzaGly-Trp-DPhe-Ala

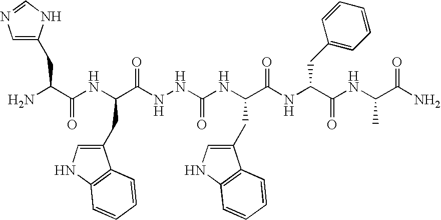

(n) HAIC-2MeDTrp-DLys-Trp-DPhe-Lys-NH₂; or

(o) ATAB-DMrp-DLys-Trp-DPhe-Lys-NH₂.

In a further embodiment, the above-mentioned peptide-like compound is Ala-AzaPhe-Ala-Trp-DPhe-Lys-NH₂ (also herein referred to as CP1A(IV) or HAIC-2MeDTrp-DLys-Trp-DPhe-Lys-NH₂ (also referred to as EP 80317; see, for example, PCT application No. PCT/EP99/08662) or ATAB-2MeDTrp-DLys-Trp-DPhe-Lys-NH₂ (also referred to as EP 80318; see, for example, PCT application No. PCT/EP99/08662).

In another embodiment, the above-mentioned selective CD36 ligand is an antibody directed against CD36, such as clone F6-A152 (Houssier M et al. Plos Med 5,2, e39, 2008).

For the method or use of the present invention, the above-mentioned selective CD36 ligand (e.g., peptide-like compound) may conveniently be presented as a pharmaceutical composition with a pharmaceutically acceptable carrier or excipient. Accordingly, the present invention provides a composition for preventing and/or treating an ischemia-related heart condition in a subject, the composition comprising a selective CD36 ligand and a pharmaceutically acceptable carrier or excipient. As used herein “pharmaceutically acceptable carrier” or “excipient” includes any and all solvents, dispersion media, coatings, antibacterial and antifungal agents, isotonic and absorption delaying agents and the like that are physiologically compatible. Alternatively, the carrier can be suitable for intravenous, intraperitoneal, subcutaneous, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art (see, for example, Rowe et al., Handbook of pharmaceutical excipients, 2003, 4^{th} edition, Pharmaceutical Press, London UK).

In an embodiment, such compositions include the selective CD36 ligand, in a therapeutically or prophylactically effective amount sufficient to prevent and/or treat an ischemia-related heart condition (e.g., myocardial I/R injury), and a pharmaceutically acceptable carrier or excipient. A “therapeutically effective amount” refers to an amount effective, at dosages and for periods of time necessary, to achieve the desired therapeutic result, such as an amelioration of symptoms or effects of an ischemia-related heart condition (e.g., (a) decreasing plasma nonesterified free fatty acids (NEFA) levels; (b) decreasing infarct size; (c) reducing myocardial NEFA uptake; (d) decreasing myocardial oxidative metabolism; (e) decreasing myocardial blood flow; (f) increasing end-diastolic and end-systolic ventricular volumes; (g) increasing stroke volume; (h) increasing the relative ratio of phosphorylated Akt to total Akt in myocardial cells; (i) increasing (transiently) the relative ratio of phosphorylated AMPK to total AMPK in myocardial cells; (j) decreasing myocardial leukocyte accumulation and/or (k) decreasing circulating blood leukocyte activation. A therapeutically effective amount of a selective CD36 ligand may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. A “prophylactically effective amount” refers to an amount effective, at dosages and for periods of time necessary, to achieve the desired prophylactic result, such as preventing or inhibiting an ischemia-related heart condition. A prophylactically effective amount can be determined as described above for the therapeutically effective amount. For any particular subject, specific dosage regimens may be adjusted over time according to the individual need and the professional judgment of the person administering or supervising the administration of the compositions.

In an embodiment, the above-mentioned prevention/treatment is mediated by a combination of at least two active/therapeutic agents. Thus, the pharmaceutical compounds of the present invention (a selective CD36 ligand) may be administered alone or in combination with other active agents useful for the treatment, prophylaxis or amelioration of symptoms of an ischemia-related heart condition. The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time.

In an embodiment, the above-mentioned selective CD36 ligand or composition comprising same is administered before the onset of ischemia and/or reperfusion. In another embodiment, the above-mentioned selective CD36 ligand or composition comprising same is administered at the onset and/or during ischemia and/or reperfusion.

In another aspect, the present invention provides a kit or package comprising at least one of the above-mentioned selective CD36 ligand (or a pharmaceutical composition comprising the selective CD36 ligand) together with instructions for its use for the prevention and/or treatment of an ischemic-related heart condition in a subject. The kit may further comprise, for example, containers, buffers, a device (e.g., syringe) for administering the selective CD36 ligand or a composition comprising same.

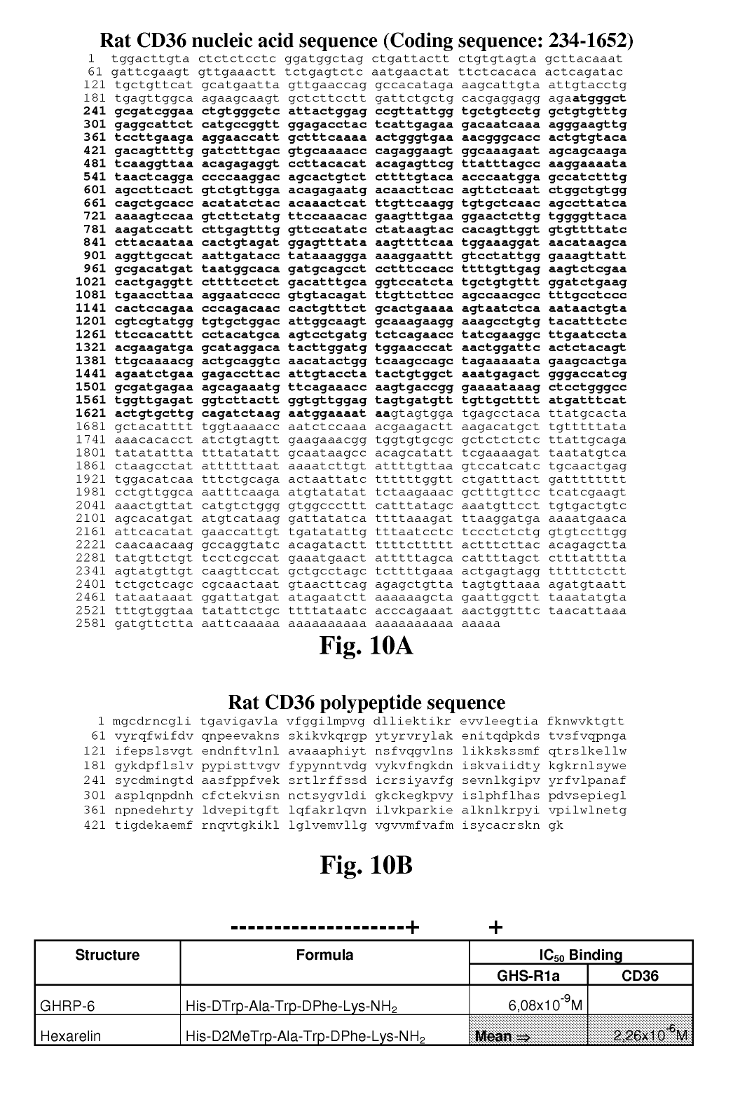

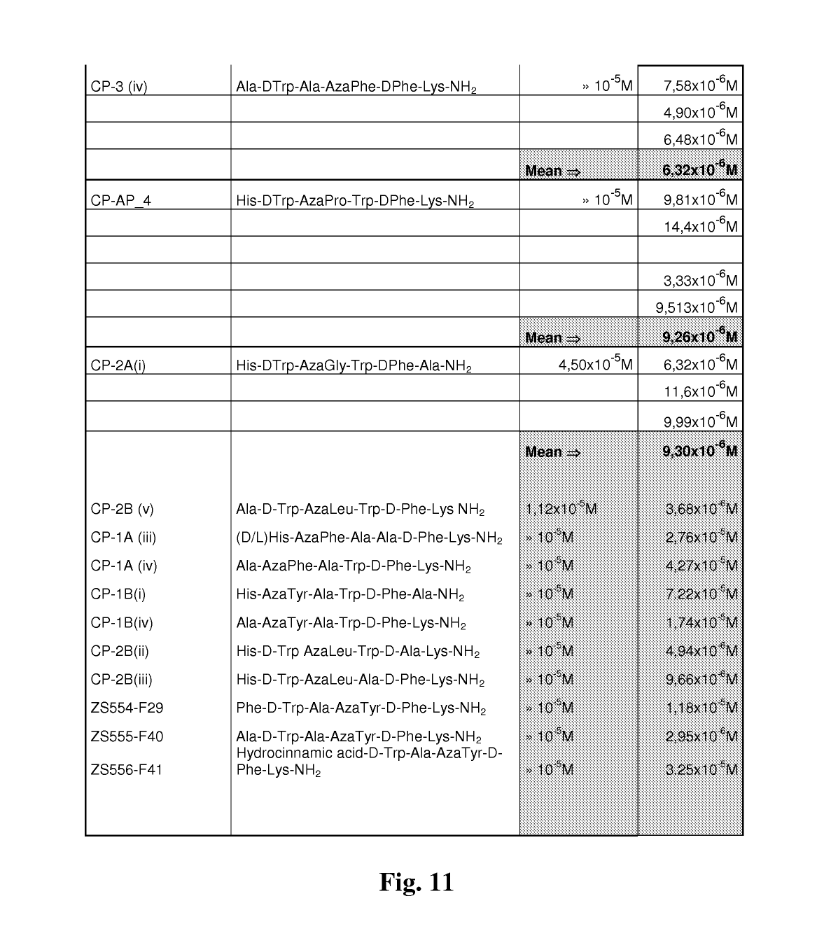

In another aspect, the present invention provides a method for identifying a compound, or determining whether a test compound may be useful, for preventing and/or treating an ischemia-related heart condition (e.g., myocardial I/R injury), said method comprising determining the binding of said compound to CD36 (e.g., a CD36 polypeptide or a fragment thereof), wherein the binding of said compound to CD36 is indicative that said compound may be useful for preventing and/or treating said ischemia-related heart condition. In an embodiment, the above-mentioned CD36 polypeptide or fragment thereof comprises a region corresponding to residues 132 to 177 (Asn¹³²-Glu¹⁷⁷) of the rat heart CD36 polypeptide (FIG. 10B). In a further embodiment, the above-mentioned CD36 polypeptide or fragment thereof comprises a region encompassing a residue corresponding to residue 169 (Met¹⁶⁹) of the rat heart CD36 polypeptide.

In an embodiment, the above-mentioned method further comprises determining whether said compound binds to a growth hormone secretagogue receptor (e.g., GHS-R1a). Lower than normal levels of binding to a ghrelin receptor (i.e., relative to a native GHS-R1a ligand) or no or substantially no binding to a GHS receptor is further indicative that a candidate compound may be useful for preventing and/or treating said ischemia-related heart condition.

Methods to measure the binding of a compound to CD36 and/or to a GHRH receptor are well known in the art (see, for example, WO 08/154738).

In another aspect, the present invention provides a method for determining whether a test compound may be useful for preventing and/or treating an ischemia-related heart condition (e.g., myocardial I/R), said method comprising contacting said test compound with a cell expressing CD36, and measuring a CD36-associated activity, wherein a modulation of said CD36-associated activity in the presence of said test compound (relative to the absence thereof) is indicative that said test compound may be useful for preventing and/or treating said ischemia-related heart condition.

In an embodiment, the above-mentioned method further comprises determining whether said compound modulates a GHS-related activity (e.g., a binding activity to a GHS receptor).

In an embodiment, the above-mentioned CD36-associated activity is a CD36-binding activity. In another embodiment, the above-mentioned CD36-associated activity is a biological activity associated with CD36.

In another embodiment, the above-mentioned CD36-associated activity is a modulation (e.g., activation) of a signaling pathway associated with CD36, such as the PI3K/Akt pathway (e.g., a modulation of the phosphorylation status of a member of this pathway such as Akt). In a further embodiment, the above-mentioned CD36-associated activity is determined based on the ratio of phosphorylated Akt to total Akt.

In another embodiment, the above-mentioned CD36-associated activity is a modulation (e.g., activation) of the AMPK pathway (e.g., a modulation of the phosphorylation status of a member of this pathway such as AMPK). In a further embodiment, the above-mentioned CD36-associated activity is determined based on the ratio of phosphorylated AMPK to total AMPK.

In an embodiment, the above-mentioned modulation of a CD36-associated activity in the presence of said test compound is a decrease in the phosphorylation of the serine/threonine protein kinase Akt/PKB (Akt) (e.g., a decrease in the ratio of phosphorylated Akt to total Akt) and/or a transient decrease in the phosphorylation of AMP-activated protein kinase (AMPK) (e.g., a decrease in the ratio of phosphorylated AMPK to total AMPK).

The above-noted assays may be applied to a single test compound or to a plurality or “library” of such compounds (e.g., a combinatorial library). Any such compounds may be utilized as lead compounds and further modified to improve their therapeutic, prophylactic and/or pharmacological properties preventing and/or treating an ischemia-related heart condition.

Test compounds (drug candidates) may be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Screening assay systems may comprise a variety of means to enable and optimize useful assay conditions. Such means may include but are not limited to: suitable buffer solutions, for example, for the control of pH and ionic strength and to provide any necessary components for optimal activity and stability (e.g., protease inhibitors), temperature control means for optimal activity and or stability, of CD36, and detection means to enable the detection of its activity. A variety of such detection means may be used, including but not limited to one or a combination of the following: radiolabelling, antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(strept)avidin), and others.

Competitive screening assays may be done by combining a CD36 polypeptide, or a fragment thereof (a CD36 binding domain) and a probe to form a probe:CD36 binding domain complex in a first sample followed by adding a test compound. The binding of the test compound is determined, and a change, or difference in binding of the probe in the presence of the test compound indicates that the test compound capable is capable of binding to the CD36 binding domain and potentially modulating CD36 activity.

The binding of the test compound may be determined through the use of competitive binding assays. In this embodiment, the probe is labeled with an affinity label such as biotin. Under certain circumstances, there may be competitive binding between the test compound and the probe, with the probe displacing the candidate agent. In an embodiment, the test compound may be labeled. Either the test compound, or a compound of the present invention, or both, is added first to the CD36 binding domain for a time sufficient to allow binding to form a complex.

The assay may be carried out in vitro utilizing a source of CD36 which may comprise a naturally isolated or recombinantly produced CD36 (or a variant/fragment thereof), in preparations ranging from crude to pure. Such assays may be performed in an array format. In certain embodiments, one or a plurality of the assay steps are automated.

A homolog, variant and/or fragment of CD36 which retains activity (e.g., a binding activity) may also be used in the methods of the invention.

“Homology”, “homologous” and “homolog” refer to sequence similarity between two polypeptide molecules. Homology can be determined by comparing each position in the aligned sequences. A degree of homology between amino acid sequences is a function of the number of identical or matching amino acids at positions shared by the sequences. Two amino acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%, e.g., with any of the sequences described herein. As used herein, a given percentage of homology between sequences denotes the degree of sequence identity in optimally aligned sequences. An “unrelated” or “non-homologous” sequence shares less than 40% identity, though preferably less than about 25% identity, with any of the sequences described herein.

Two sequences (polypeptide or nucleotide) are considered substantially identical if, when optimally aligned, they share at least about 50% sequence identity. In alternative embodiments, sequence identity may for example be at least 60%, at least 70%, at least 75%, at least 80%, at least 85%, at least 90%, or at least 95%, e.g., with any of the sequences described herein. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48: 443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially identical is based on one of the sequences being substantially complementary to the complement of the other. In an embodiment, substantially complementary sequences are two sequences that hybridize to each other under moderately stringent, or preferably stringent, conditions. Hybridisation to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausu bel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In an embodiment, the above-mentioned homolog, variant and/or fragment of CD36 comprises a region corresponding to residues 132 to 177 (Asn¹³²-Glu¹⁷⁷) of the rat heart CD36 polypeptide (FIG. 10B). In a further embodiment, the above-mentioned CD36 polypeptide or fragment thereof comprises a region encompassing a residue corresponding to residue 169 (Met¹⁶⁹) of the rat heart CD36 polypeptide.

In an embodiment, the above-mentioned subject is an animal such as a mammal. In a further embodiment, the above-mentioned mammal is a human.

### MODE(S) FOR CARRYING OUT THE INVENTION

The present invention is illustrated in further details by the following non-limiting examples.

### Example 1

### Materials and Methods

Animals. CD36^{-/-} mice were generated by targeted homologous recombination and backcrossed six times to C57BI/6. Wild-type control littermates (CD36^{+/+}) were bred from the same cross and were therefore of identical genetic background [Febbraio et al., 2000]. Male mice, aged 23 (±1) weeks, were used for experiments. They were fed standard chow (#5075, Charles Rivers, Saint-Constant, Québec, Canada) and water ad libitum, and housed singly during treatment periods (2 or 10 weeks). Daily pharmacological treatments with 300 μg/kg of EP 80317 or CP1A(IV) or vehicle (0.9% NaCl) were done by subcutaneous (s.c.) injections.

C57BU6 mice were bred in house. Male mice, aged 23 (±1) weeks, were used for experiments. They were fed standard chow (#5075, Charles Rivers, Saint-Constant, Québec, Canada) and water ad libitum, and housed singly during treatment periods (2 weeks). Daily pharmacological treatments with 300 μg/kg (289 nmol/kg) of EP 80317 or 289 nmol/kg of CP-3(IV), 289 nmol/kg of CP-AP-4 or vehicle (0.9% NaCl) were done by subcutaneous (s.c.) injections.

Compounds. EP 80317 (HAIC-2MeDTrp-DLys-Trp-DPhe-Lys-NH₂) was synthesized as previously described (PCT application No. PCT/EP99/08662). CP1A(IV) (Ala-AzaPhe-Ala-Trp-DPhe-Lys-NH₂), CP-3(IV) (Ala-D-Trp-Ala-AzaPhe-DPhe-Lys-NH₂) and CP-AP-4 (His-D-Trp-AzaPro-Trp-DPhe-Lys-NH₂) were synthesized as previously described (PCT publication No. WO 08/154738).

Experimental model of IHD: Transient left coronary artery ligation in vivo. Mice were subjected to a transient LCAL surgery as described before, with minor modifications [Tarnayski et al., 2004]. Mice were injected i.p. with buprenorphine (0.05 mg/kg) and placed in an induction chamber with inhalation anesthesia comprised of 3% isoflurane mixed with 100% oxygen. Mice were intubated with a blunt ended 20-gauge catheter into the trachea via the mouth and mechanically ventilated at a tidal volume of 7 ml/kg at 130 respirations/min with a MiniVent™ mouse ventilator (model 845, Harvard Apparatus, Saint-Laurent, QC, Canada). Anesthesia was maintained with 1.5-2% isoflurane and the animals were kept warm using electrical heating pads during the surgical procedure. The chest was opened by a horizontal incision through the skin and muscle layers at the third intercostal space exposing the left side of the heart. The left anterior coronary artery was identified 1 mm inferior to the left atrial appendage using a stereomicroscope (SMZ645, Nikon, Mississauga, Ontario, Canada) and a 8-0 silk suture was passed underneath the artery at this point and tied over a 2-mm section of PE-10 tubing. Visual blanching distal to the coronary occlusion confirmed myocardial ischaemia. Lidocaine (6 mg/kg) was administered i.p. just following occlusion and prior to reperfusion. After 30 minutes, the PE10 tubing was removed allowing reperfusion. The lungs were reinflated and the chest wound closed layer by layer before extubation. At 6 hours following reperfusion, mice were anesthetized (isoflurane), a blood sample withdrawn, and the heart arrested in diastole by an intravenous injection of 1 M KCl (0.5 mL). Hearts were immediately frozen at −80° C. unless otherwise stated.

Evaluation of area at risk and infarct size. Two days after reperfusion, the left anterior descending (LAD) coronary artery was re-occluded at the original site and the abdominal aorta was injected retrogradly with 5% Evans blue dye to delineate the AAR by the absence of dark blue staining. The left ventricle, including the interventricular septum, was dissected and cut into transverse 1-mm slices from the apex to the base, using an acrylic matrix (Alto Inc., Hatfield, Pa., USA). The slices were incubated in 1% triphenyltetrazolium chloride solution at 37° C. for 15 min and placed in 10% neutral buffered formalin for 12 hours. Each slice was weighed and photographed on both sides with a digital camera (Nikon, Coolpix™ 4500, Mississauga, Ontario, Canada). The total LV area, AAR and IA were determined for each side of a slice by planimetric analysis using Adobe CS3 Photoshop™ software (Ottawa, ON, Canada), and averaged. The infarct weight was determined as follows: [(A1×WT1)+(A2×WT2)+(A3×WT3)+(A4×WT4) . . . ] where A and WT are the infarct area and weight of the section, respectively. The AAR weight was calculated in a similar manner, but by subtracting the blue (viable) stained zone from the weight. The results are expressed in terms of % IA/AAR, IA/LV and AAR/LV.

Imaging experiments. Imaging experiments were performed with the avalanche photodiode-based small animal PET scanner (μPET) [Lecomte et al., 1996]. Before imaging, the heart position was localized with a Doppler probe (0.64 cm, 9 MHz; Parks Medical Electronics). During imaging, the animals rested supine on the scanner bed and were kept warm with a heating pad. In one set of experiments, [¹¹C]-acetate (˜20 MBq in 0.150 ml) and [¹⁸F]-fluoro-deoxyglucose (FDG) (˜37 MBq in 0.150 ml) were used to determine myocardial oxidative metabolism (VO₂) and glucose utilization, respectively, as previously described [Menard et al., 2009]. In another set of experiments, [¹⁸F]-fluoro-thia-6-heptadecanoic acid (FTHA) (˜37 MBq in 0.150 ml) were used to determine NEFA uptake as previously described [Ci et al., 2006]. In a previous study, we have demonstrated that FTHA is a good marker of total and mitochondrial NEFA uptake in the myocardium of rats during normoinsulinemic and hyperinsulinemic conditions as compared to [¹⁴C]-bromopalmitate and [¹⁴C]-palmitate [Ci et al., 2006]. List-mode dynamic acquisitions were performed for all tracers and additional EKG-gated dynamic acquisitions were performed with FTHA and FDG. Blood samples were taken at the end to determine blood glucose, plasma insulin, NEFA and TG levels [Menard et al., 2009].

Imaging Data Analysis. For [¹¹C]-acetate images, dynamic series of 25 frames each were sorted out whereas 27 frames were used for ¹⁸FDG and ¹⁸FTHA imaging, and were reconstructed and analyzed using multicompartmental analysis [Menard et al., 2009] or the Patlak method for FTHA [Patlak and Blasberg, 1985]. For analysis of ventricular function, PET data from FTHA and FDG images were obtained as a series of 8 ECG-gated frames and were reconstructed as a series of adjacent 2-dimensional slice using 20 iterations of the maximum-likelihood expectation maximization algorithm. The Corridor4DM™ v5.2 software (Segami, Invia LLC, MI, USA) was used for reorientation and to compute left ventricular volumes (LW) and left ventricular ejection fraction (LEVF) after validation with small rodent heart phantoms, as described previously [Croteau et al., 2003].

Plasma and tissue assays. Plasma insulin, triglyceride (TG) and NEFA levels were measured as previously described [Menard et al., 2009]. In vitro lipolysis was prevented by collecting blood in the presence of 40 mM Orlistat (Calbiochem, San Diego, Calif., USA) and centrifuging rapidly. Plasma and tissue lipids were extracted according to the method described by Folch et al. [Folch et al., 1957]. The non-metabolized fraction of [¹⁸F]-FTHA in plasma was determined using thin-layer chromatography from blood samples taken 2, 3, 5, 10, and 30 min after FTHA injection, and the metabolite corrected plasma curve was calculated by linear interpolation and used to correct the plasma input function (modified from [Ci et al., 2006]). Myocardial mitochondria were extracted with measurement of GDH activity to correct for extraction efficiency [Menard et al., 2009].

Western blot analysis. Total ventricular protein lysates were prepared as described previously [Bodart et al., 2002]. Left ventricles were homogenized in PBS containing a protease inhibitor cocktail (Roche Applied Science, Indianapolis, Ind., USA) and 1 mM sodium orthovanadate. Homogenates were incubated for 10 min on ice with an equal volume of lysis buffer (NaCl 300 mM, Tris-HCl 100 mM, 2% Triton X-100, 0.2% SDS, 50 mM NaF, 4 mM EDTA, 1 mM sodium orthovanadate and protein inhibitors, pH=7.5), and centrifuged at 14,000 g for 30 minutes at 4° C. The protein concentration of the su pernatant was determined by the bicinchoninic acid (BCA) protein assay (Pierce Biotechnology, Rockford, Ill., USA). Equal amounts (50 or 100 μg) of protein extracts were separated on 10% SDS-polyacrylamide gels and transferred electrophoretically to polyvinylidene difluoride (PVDF) membranes (Bio-Rad Laboratories, Hercules, Calif., USA) for immunoblotting. Membranes were incubated 1 h at room temperature with 5% BSA in TBS (150 mM NaCl and 10 mM Tris-HCl, pH=7.6) containing 0.05% Tween™ 20, washed briefly in TBS and incubated overnight at 4° C. with anti-Akt (#9272, diluted 1:1000), anti-phospho-Akt (Ser473) (#9271, diluted, 1:1000), anti-AMPKα (which recognizes both α1- and α2-subunits) (#2532, diluted 1:1000), anti-phospho-AMPK (Thr172) (#2531, diluted 1:1000), all primary rabbit antibodies were purchased from New England Biolabs (Beverly, Mass., USA) and anti-mouse α-tubulin (#ab7291, diluted 1:1000) from Abcam (Cambridge, Mass., USA). After washing steps, blots were incubated for 1 h at room temperature with horseradish peroxidase-conjugated secondary goat anti-rabbit IgG (#111-035-008, diluted 1:5000) from Jackson lmmunoresearch (West Grove, Pa., USA), except for anti-α-tubulin, for which secondary goat anti-mouse IgG was used (#074-1806, diluted 1:5000) (KPL, Gaithersburg, Md., USA). Antibody binding was detected by enhanced chemiluminescence using an alpha Imager™ (Alpha Innotech Corporation, San Leandro, Calif., USA). Quantification of the digital images obtained was performed using ImageQuant™ 5.2 software (Molecular Dynamics, Sunnyvale, Calif., USA).

Myeloperoxidase assay. Myocardial myeloperoxidase (MPO) activity was assayed as previously described [Belanger et al., 2008], with some modifications. Briefly, whole left ventricles were homogenized in 500 μl PBS and the pellets were homogenized in 350 μl acetate buffer (100 mM), pH 6.0, containing 1% hexadecyltrimethylammonium bromide and 20 mM EDTA. Left ventricular homogenates were heated to 65° C. for 120 min in a water bath. The homogenates were subjected to three freeze-thaw cycles and then centrifuged at 2,000 g for 10 minutes. MPO was assayed by incubating supernatants with 3.2 mM 3,3′,5,5′-trimethylbenzidine and 0.3 mM H₂O₂ for 5 min at 37° C. The reaction was stopped by the adding 0.2 M sodium acetate (pH 3.0). Polymorphonuclear leukocytes (PMN) calibration curves were prepared using peritoneal mouse PMN (elicited by an i.p. injection of 2 ml per mouse of a 5% casein solution in saline) and purified using magnetic cell separation (MACS, Miitenyi Biotec, Auburnm; Calif., USA) with magnetic microbeads conjugated to Ly-6G highly expressed on neutrophils, according to the manufacture's instructions. The numbers of PMN per left ventricle were calculated from the standard curves.

Whole blood chemiluminescence. Luminol-enhanced whole blood chemiluminescence of mouse leukocytes was studied using opsonized zymosan (10 mg/ml) as a stimulus. Briefly, heparinized blood was collected and processed immediately after diluting (1/10) in DMEM containing 50 mM HEPES and 1 mM luminol. The chemiluminescence signals were recorded using a computer-assisted luminometer (model 500; Chronolog Corp, Havertown, Pa., USA). Chemiluminescence intensities were measured as the peak amplitude in arbitrary units.

Reactive oxygen species in whole heart homogenate. Determination of NADPH oxidase activity was evaluated in left ventricle. Briefly, the tissue was rinsed in PBS and homogenized 3 times in ice-cold Krebs buffer for 15 sec, using a PowerGen™ 700 homogenizer (Fisher Scientific Canada). Homogenates were then incubated at 37° C. for 5 min in the presence of lucigenin (final concentration 5 μM). Lucigenin-enhanced chemiluminescence was assessed to determine O₂ after adding NADPH (300 μM), in the presence or absence of DPI (300 μM), a NADPH oxidase inhibitor. The chemiluminescence signals were recorded using a computer-assisted luminometer (Chronolog Corp. Havertown, Pa., USA). Chemiluminescence intensities were measured as the peak amplitude and were corrected for protein concentration.

Protein concentration in whole heart homogenate. Protein levels in heart homogenates were determined using the BCA Protein Assay, Thermo Scientific (Rockford, Ill., USA) according to the manufacturer's instructions.

Cardiac troponin I (cTnI) concentrations in plasma. cTnI plasma levels were assayed using the High Sensitivity Mouse Cardiac Troponin-I ELISA Kit (Life Diagnostics, Inc., West Chester, Pa., Cat. No. 2010-1-HSP), according to the manufacturer's instructions.

Blood lactate. Blood lactate was determined using the Lactate Pro Analyser (FaCT Canada Consulting Ltd, Quesnel, BC, Canada), according to the manufacturer's instructions.

Systemic blood pressure. Systemic blood pressure was assessed by using a fluid-filled, heparinized catheter inserted into the left carotid artery. Pressures waveforms were collected (at 1 kHz) and analyzed by using a 16-channel data acquisition and software system (IOX; EMKA Technologies, Falls Church, Va). Data were acquired from 5h45 post-ischemia and averaged over a period of 15 minutes.

Statistical analysis. Data are expressed as mean±S.E. Comparisons between groups were performed using unpaired t test or a one- or two-way ANOVA, where appropriate, followed by pair-wise multiple comparisons using Student-Newman-Keuls post-hoc test (GraphPad Prism™ Software, La Jolla, Calif., USA). Differences were considered significant at P<0.05.

### Example 2

### Effect of EP 80317 on Body and Left Ventricular Weights and Plasma Lipid Profiles

On average, CD36^{-/-} mice did not show lower body weight (BW) than aged-matched, CD36^{+/+} control littermates, however left ventricular (LV) weights were higher (Table I). Mean LV/BW ratio was slightly increased in CD36^{-/-} mice, indicating modest LV hypertrophy (Table I) [Irie et al., 2003; Yang et al., 2007]. EP 80317 did not modulate BW or LV/BW ratio.

TABLE 1
Body weights and left ventricular weights/body
weights in CD36^{-/-} and CD36^{+/+} 48 hours
after transient myocardial ischemia-reperfusion

| | Body wt | LV wt | LV wt/body wt |
|---|---|---|---|
| Genotype | (g) | (g) | (mg/g) |
| CD36^{+/+} | 26.4 ± 2.1 | 0.084 ± 0.006 | 3.2 ± 0.1 |
| CD36^{-/-} | 26.6 ± 0.4 | 0.102 ± 0.003* | 3.8 ± 0.1*** |

Age-matched CD36^{+/+} (n = 8) and CD36^{-/-} (n = 12) male mice 48 hours after LCAL surgery.
Values are mean ± S.E..
*p < 0.05,
***p < 0.001 compared to 0.9% NaCl control.

Plasma NEFA concentrations were transiently elevated following LCAL ligation in non-fasted mice, whether the mice were deficient in CD36 or not (Table II). EP 80317 treatment attenuated plasma NEFA elevation by 29% (p<0.05) in a CD36-dependent manner (Table II). Hence, EP 80317, by reducing the circulating NEFA to which the heart is exposed, will lead to reduced cardiac fatty acid oxidation, which has protective effects in cardiac ischemia. In contrast to the reduction in plasma NEFA levels observed in EP 80317-treated mice 6 hours after reperfusion, no effect of the peptide was observed at 48 hours, when plasma NEFA were back to control levels (Table II). Plasma NEFA levels were nearly twice as elevated in CD36^{-/-} mice as compared to their CD36^{+/+} counterparts after 48 hours reperfusion, whether treated or not with EP 80317 (Table II). Total plasma cholesterol was slightly increased in CD36^{-/-} compared to the control littermates as reported before, the latter attributed to a rise of HDL cholesterol in CD36-deficient mice [Brundert et al., 2006] (Table II). EP 80317 did not modulate plasma TG at either 6 or 48 hours following reperfusion in both CD36^{+/+} and CD36^{-/-} mice.

TABLE II
Plasma cholesterol and triglycerides profile of CD36^{-/-} mice and their control C57BL/6
wild type littermates 6 or 48 hours after myocardial ischaemia-reperfusion in mice treated or not
with EP 80317 or vehicle.

| | | | | | | Glycemia | Glycemia |
|---|---|---|---|---|---|---|---|
| H Post- | Geno- | | Total | | | Before | Post- |
| Reperfusion | type | Tx | Cholesterol | Triglyceride | NEFA | Ischemia | Reperfusion |
| 6 h | CD36^{+/+} | 0.9% | 1.4 ± 0.1 (8) | 0.44 ± 0.03 | 0.49 ± | 11.2 ± 0.5 (19) | 13.5 ± 0.8 (22) |
| | | NaCl | | (8) | 0.04 (4) | | |
| | | EP | 1.4 ± 0.3 (4) | 0.35 ± 0.04 | 0.35 ± | 10.6 ± 0.4 (15) | 13.6 ± 1.2 (20) |
| | | 80317 | | (4) | 0.02* (5) | | |
| | CD36^{-/-} | 0.9% | 1.8 ± 0.1 (6) | 0.62 ± 0.10 | 0.42 ± | 9.5 ± 0.3 (8) | 8.8 ± 0.6 (15)* |
| | | NaCl | | (6) | 0.03 (6) | | |
| | | EP | 1.9 ± 0.1 (5) | 0.58 ± 0.16 | 0.44 ± | 9.3 ± 0.5 (8) | 8.8 ± 0.6 (17)* |
| | | 80317 | | (5) | 0.04 (5) | | |
| 48 h | CD36^{+/+} | 0.9% | 1.9 ± 0.2 (6) | 0.53 ± 0.08 | 0.11 ± | — | — |
| | | NaCl | | (6) | 0.01 (5) | | |
| | | EP | 1.5 ± 0.03 (6) | 0.44 ± 0.03 | 0.10 ± | — | — |
| | | 80317 | | (6) | 0.02 (6) | | |
| | CD36^{-/-} | 0.9% | 2.4 ± 0.1 (4)* | 0.57 ± 0.08 | 0.24 ± | — | — |
| | | NaCl | | (6) | 0.04^{##} (10) | | |
| | | EP | 2.5 ± 0.2 (4)* | 0.55 ± 0.06 | 0.19 ± | — | — |
| | | 80317 | | (5) | 0.02 (9) | | |

Age-matched CD36^{+/+} and CD36^{-/-} male mice were treated with EP 80317 (300 μg/kg/day) or 0.9% NaCl for 2 weeks.
Plasma total cholesterol, Triglyceride, Nonesterified Free Fatty Acid (NEFA) and blood glycemia values are expressed in mmol/L.
Values are mean ± S.E.M.
*p < 0.05 compared to 0.9% NaCl control;
^{#}p < 0.05;
^{##}p < 0.01 CD36^{-/-} compared to CD36^{+/+}

### Example 3

### Effect of EP 80317 on Infarct Size 48 Hours Following Transient Left Coronary Artery Ligation Surgery in CD36^{+/+} and CD36⁴^{-/-} Mice

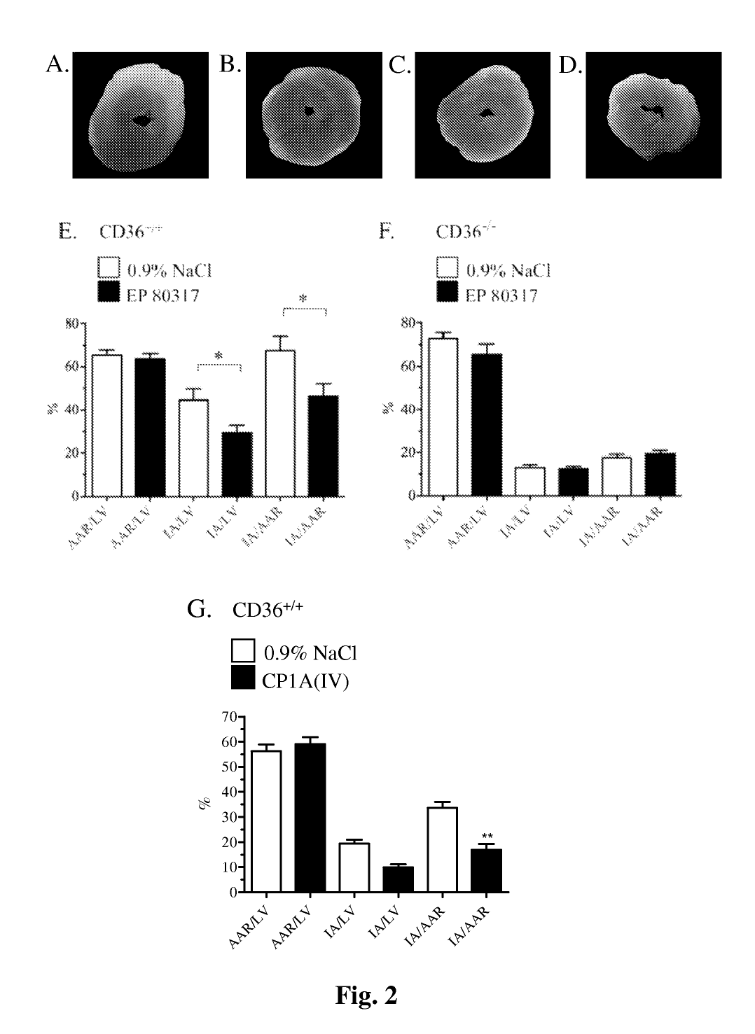

Transient LCAL caused a consistently large area-at-risk that did not differ between CD36^{+/+} (65±2%) and CD36^{-/-} mice (73±3%). Pretreatment with EP 80317 for 14 days did not modulate the AAR/LV in both CD36^{+/+} and CD36^{-/-} mice (FIG. 2E, F). However, the infarct size, as assessed by the infarct area to area-at-risk (IA/AAR) and the infarct area to left ventricular (IA/LV) surface ratios, was smaller in CD36^{-/-} (18±1% and 13±1%, respectively) than in CD36^{+/+} (68±6% and 45±5%, respectively) in vehicle-treated mice (FIG. 2E, F) (p<0.001). A 2-week treatment with EP 80317 reduced the IA/AAR ratio by 31% (p<0.05) and the IA/LV ratio by 34% (p<0.05) in CD36^{+/+} mice (FIG. 2A). In contrast, EP 80317 did not modulate infarct size in CD36^{-/-} mice (FIG. 2F). A similar reduction in infarct area was observed in CD36^{+/+} mice treated with the peptide for longer periods (10 weeks) (not shown). In addition, a 2-week pretreatment with CP1A(IV), using the same drug regimen, reduced infarct area by 49% (p<0.01%) (FIG. 2G).

### Example 4

### Effect of EP 80317 Pretreatment on ¹⁸F-FTHA Kinetics

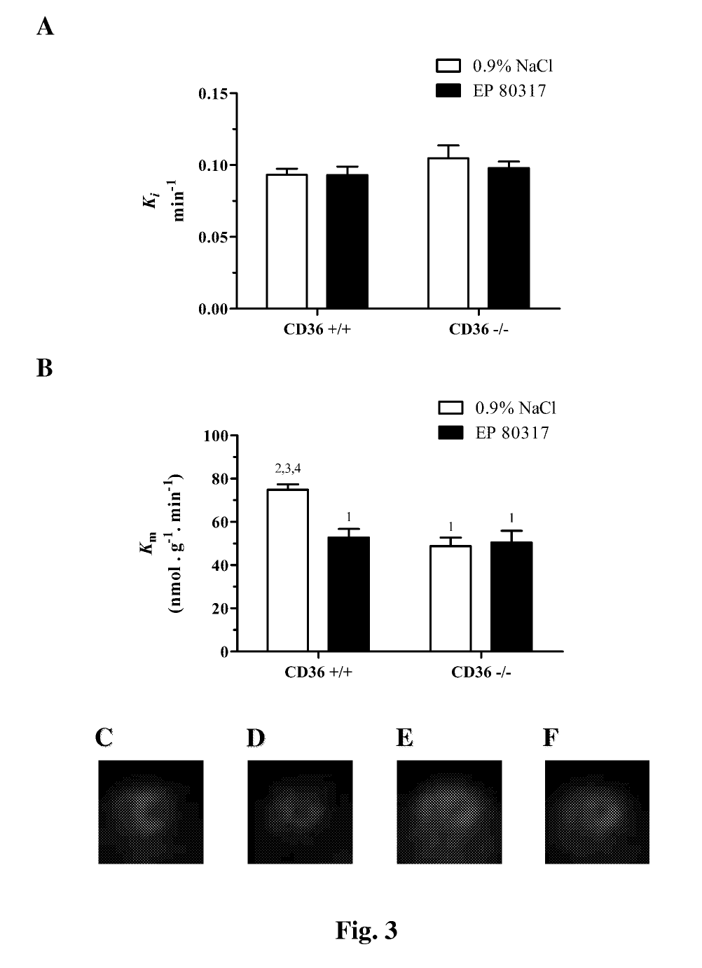

The mean fractional uptake rate (Kᵢ) derived from Patlak analysis was not affected by EP 80317, neither in CD36^{+/+} or in CD36^{-/-} mice, after 6 hours reperfusion following LCAL surgery (FIG. 3A). In addition, a similar entry rate of the fatty acid tracer in CD36^{+/+} and CD36^{-/-} mice was observed, suggesting that the expression of this scavenger receptor is not the limiting step involved in myocardial LCFA substrate uptake. The total plasma ¹⁸F activity vs. time curve was not significantly different between CD36^{+/+} vs. CD36^{-/-} mice. However, EP 80317 pre-treatment was associated with reduced total plasma NEFA uptake (Kₘ) in CD36^{+/+} mice, to the level of that observed in CD36^{-/-} mice. Without being bound to a particular theory, these observations suggest that whereas EP 80317 does not appear to modulate fractional fatty acid uptake of the heart, the net cardiac uptake of fatty acids upon treatment with the peptide is reduced, most probably as a result of reduced substrate availability. CD36^{-/-} mice show reduced net fatty acid uptake, and this effect was not modulated by EP 80317. Overall, these results support that low plasma NEFA concentrations drive the reduced myocardial plasma NEFA uptake, in a CD36-dependent manner in wild-type mice, inasmuch as EP 80317 does not further reduce Kₘ in CD36-deficient mice.

### Example 5

### Effect of EP 80317 Pretreatment on Myocardial Metabolic Rate of Glucose

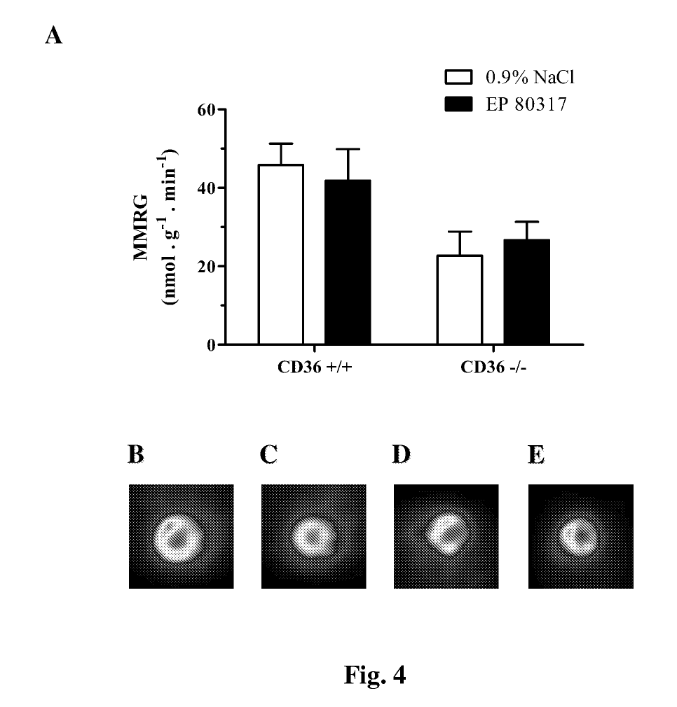

Myocardial I/R is associated with initial catecholamine discharge which mobilize fatty acid from adipose tissue, acutely inhibits insulin release from the pancreas, and elicit hyperglycemia [Opie, 2008]. In agreement, myocardial ischemia-reperfusion in mice was associated with an increase in glycemia after 6 hours reperfusion (Table II). Yet, myocardial glucose utilization, as assessed by calculating the myocardial metabolic rate of glucose (MMRG) was not modulated by EP 80317 treatment (FIG. 4). MMRG tended to be lower in CD36-deficient mice (FIG. 4).

### Example 6

### Effect of EP 80317 Pretreatment on Myocardial Blood Flow and Oxidative Metabolism

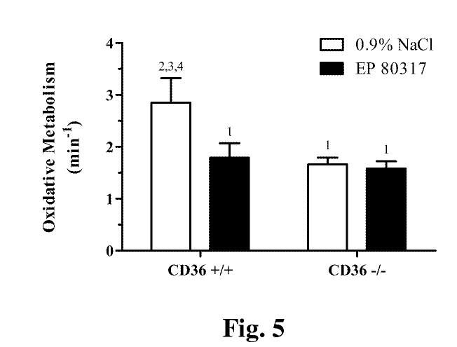

As shown in FIG. 5, myocardial oxidative metabolism was reduced in mice pre-treated with EP 80317. CD36^{-/-} mice have impaired myocardial metabolism which was unaffected by EP 80317 (FIG. 5A). Hence, the cardioprotective effect of EP 80317 appears to be linked to a reduced oxidative burst upon reperfusion.

### Example 7

### Effect of EP 80317 Pretreatment on Intracardiac Ventricular and Ejection Volumes, Ejection Fraction and Stroke Volume

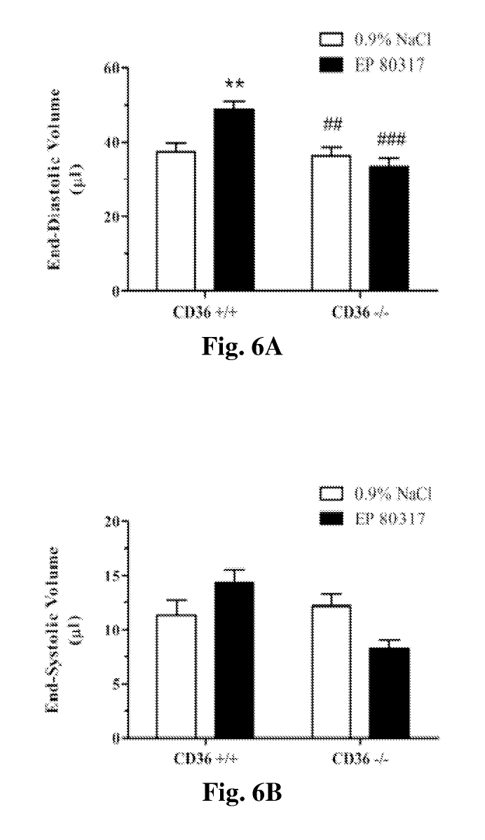

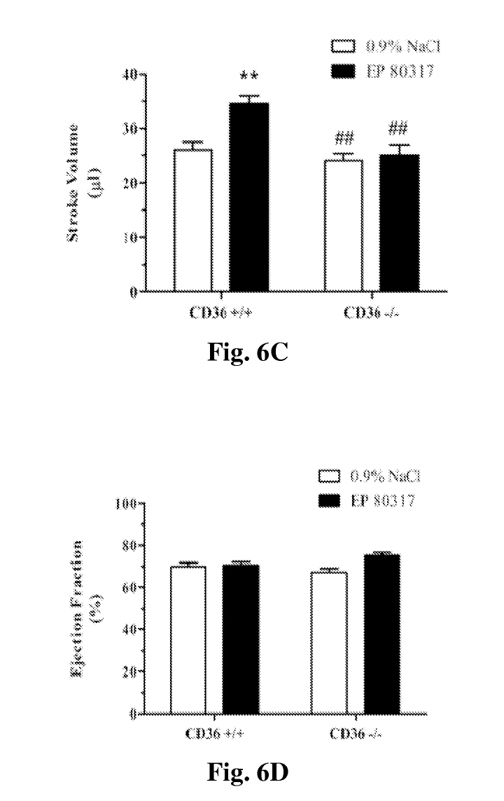

As shown in FIGS. 6A and 6B, both end-diastolic and end-systolic ventricular volumes were increased by 31% (p<0.01) and 26%, respectively, in EP 80317-treated mice. Similarly, as depicted in FIG. 6C, the stroke volume was increased by 33% (p<0.01), indicating that cardiac parameters were preserved in these mice.

### Example 8

### Effect of EP 80317 on AMPK and Akt Phosphorylation Following Transient LCAL Surgery in CD36^{+/+} and CD36^{-/-} mice

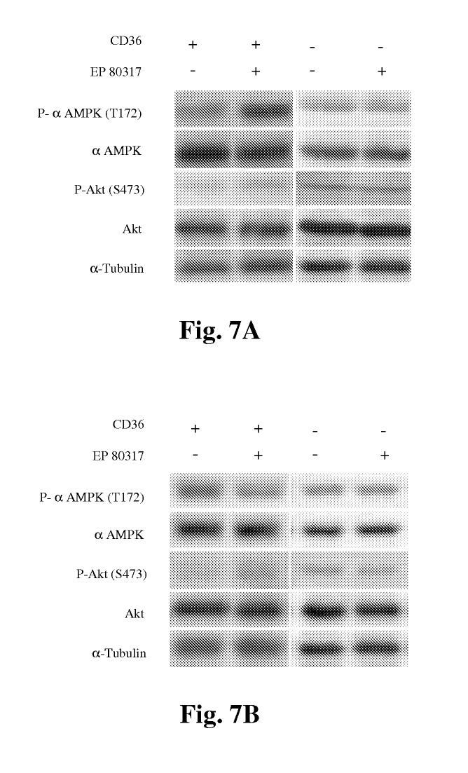

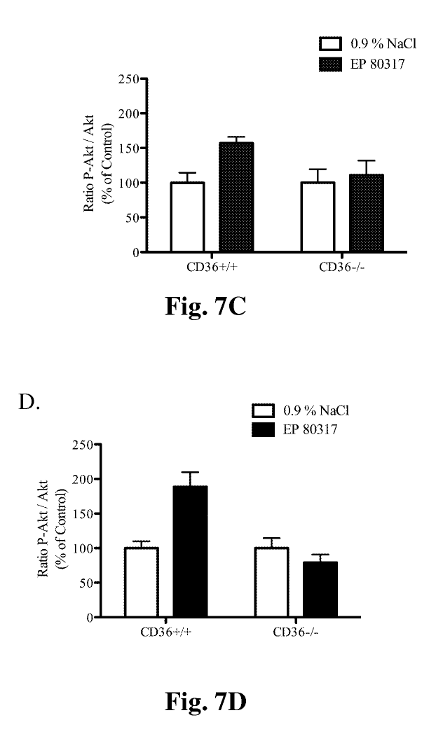

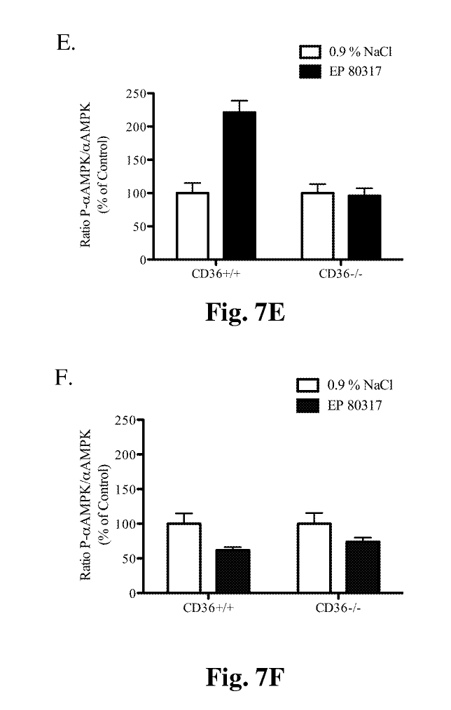

The relative ratio of phosphorylated Akt (P-Akt) to total Akt band density was increased by 57% (p<0.01) and that of phosphorylated AMPK (P-AMPK) to total AMPK by 121% (p<0.01) after 6 hours reperfusion in EP 80317-treated CD36^{+/+} mice (FIG. 7A). In contrast, no effect of the peptide was observed on either P-Akt/Akt or P-AMPK/AMPK ratios in CD36^{-/-} mice (FIG. 7B). After 48 hours of reperfusion, the density ratio of P-Akt/Akt was still increased by 89% (p<0.01) in CD36^{+/+} mice treated with EP 80317, while that of P-AMPK/AMPK tended to decrease (FIG. 7C). As observed at 6 hours, no significant effect of the peptide was observed on Akt and AMPK phosphoprotein signals (FIG. 7D).

The results show increased Akt phosphorylation in EP 80317-treated mice at 6 hours post-reperfusion, and the relative Ser(P)473-Akt to total Akt ratio was further elevated at 48 hours, in contrast to reduced AMPK phosphorylation at this late time point (FIG. 7C). This is particularly interesting considering the ability of Akt (Akt1 and 2) to negatively regulate AMPK activity through phosphorylation of AMPK (both α₁ and α₂) at Ser^{485/491}, thereby preventing its phosphorylation (and activation) at Thr¹⁷² [Kovacic et al., 2003; Soltys et al., 2006]. These observations support a regulatory role of Akt in the context of myocardial I/R which, in addition to recruiting anti-apoptotic pathways, may protect the heart from reperfusion injury as a result of decreased AMPK activity. Hence, despite some commonalities in the downstream targets of Akt and AMPK, they may also play distinct roles along the sequence of events associated with myocardial ischemia and reperfusion.

### Example 9

### Effect of EP 80317 Pretreatment on Myocardial Leukocyte Activation and Accumulation After 48 Hours Reperfusion Following LCAL Surgery in CD36^{+/+} and CD36^{-/-} Mice

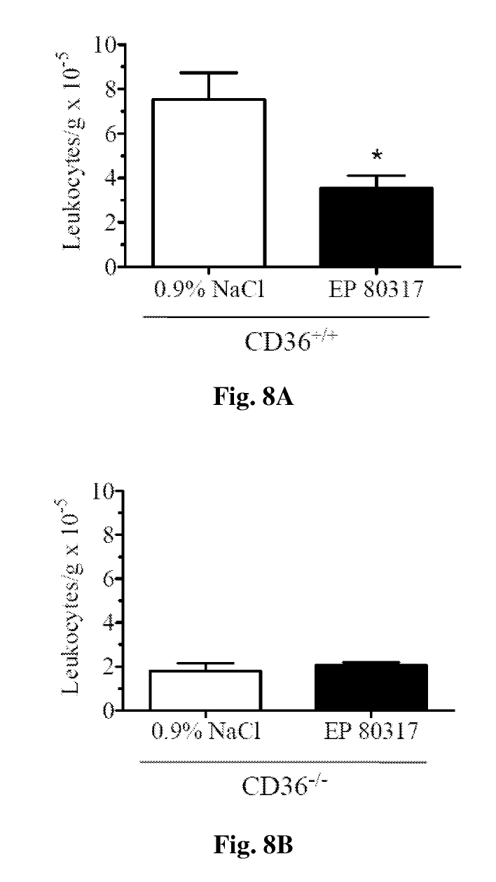

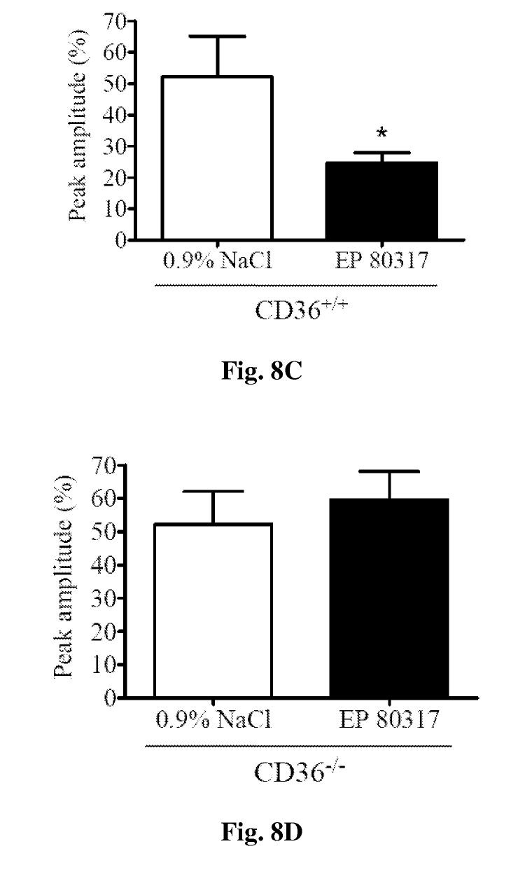

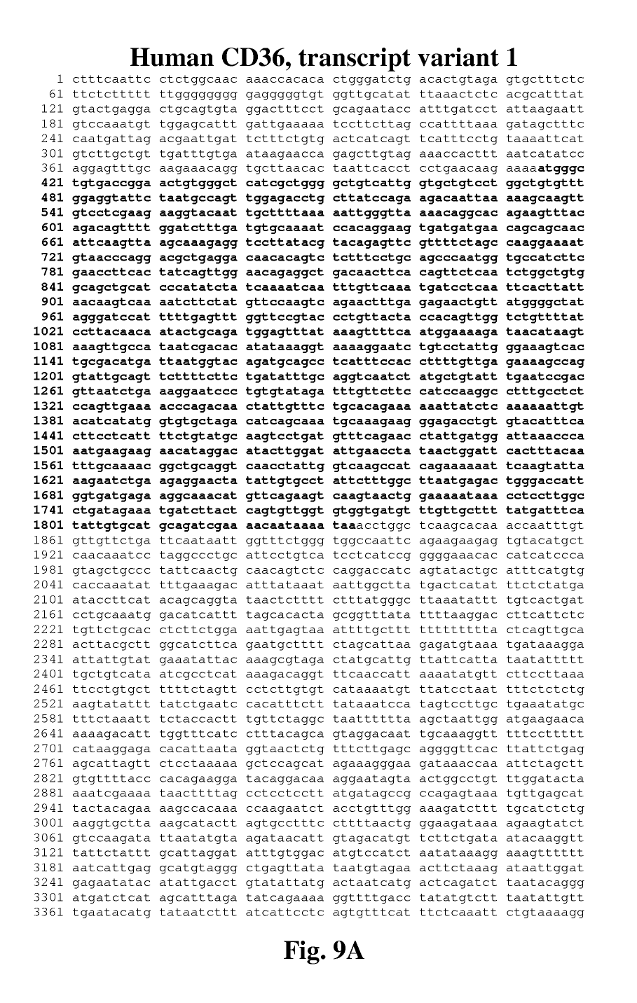

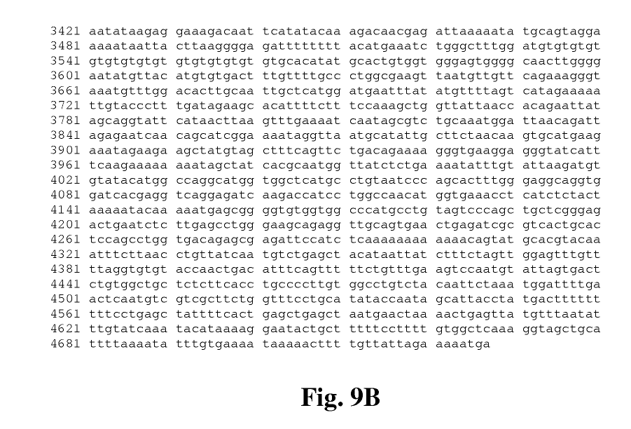

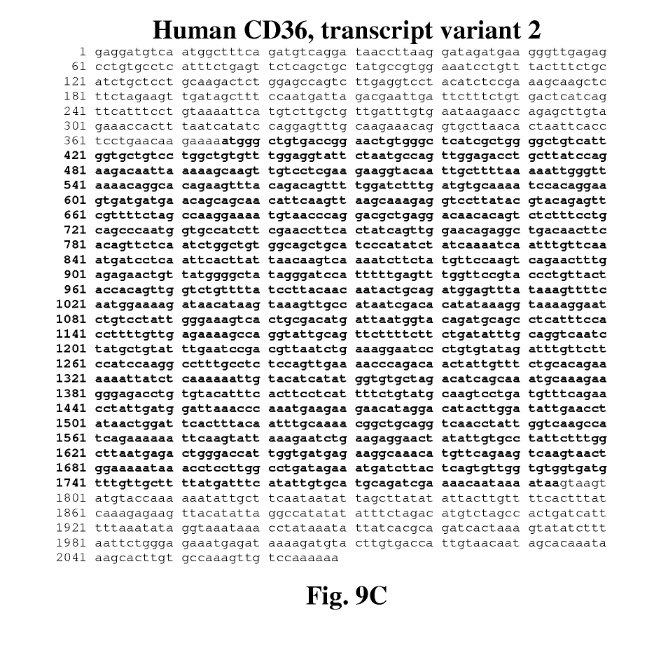

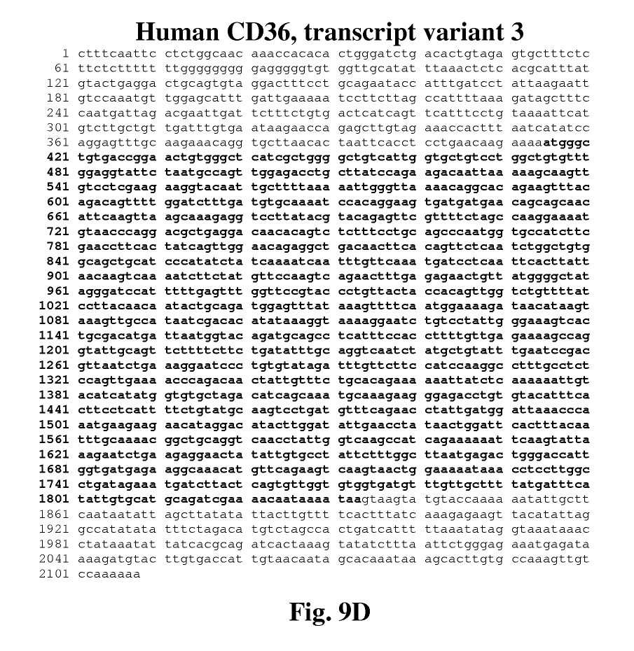

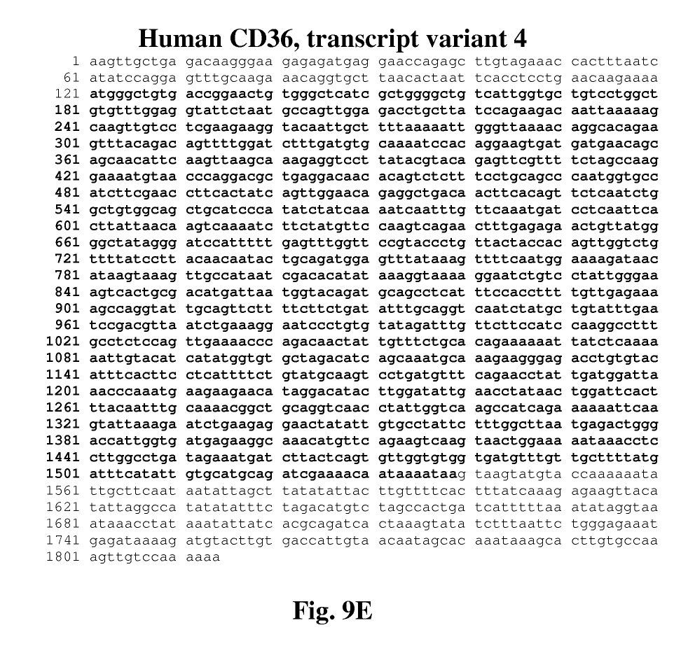

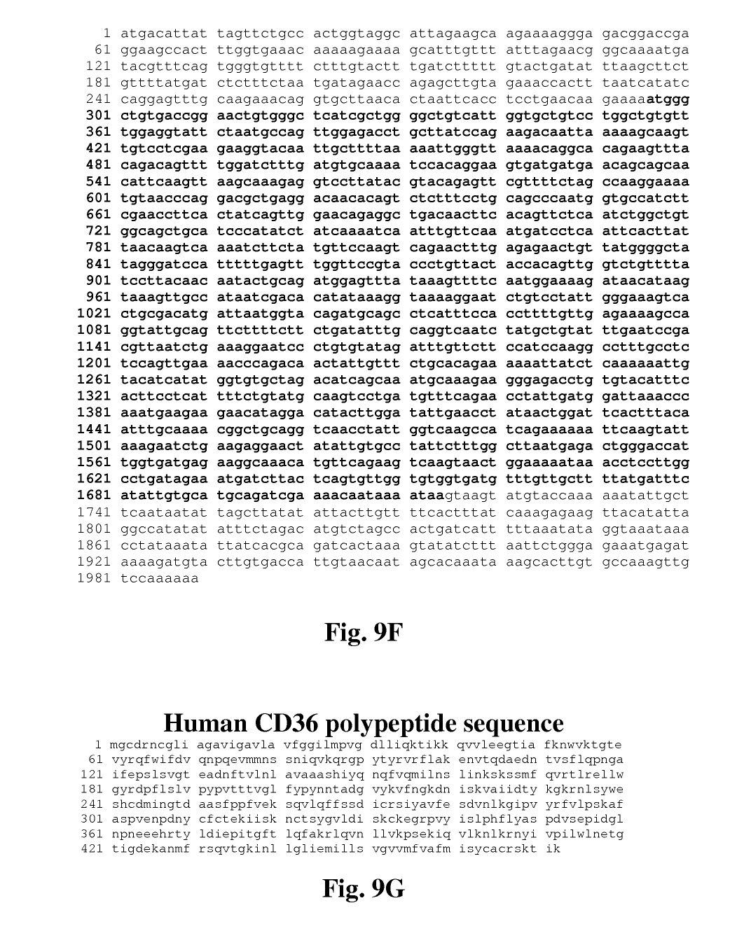

EP 80317 pretreatment was associated with a CD36-dependent, 53% (p<0.05) reduction in myocardial leukocyte accumulation after 48 hours reperfusion (FIGS. 8A and 8B). Circulating blood leukocyte priming and/or activation was also reduced by 53% in (p<0.05) in EP 80317-treated CD36^{+/+} mice, as assessed by opsonized zymosan-induced and luminol-enhanced chemiluminescence (FIG. 8C), in contrast to blood harvested from CD36-deficient mice (FIG. 8D). These observations support that EP 80317 may reduce myocardial tissue injury and pathological remodeling following reperfusion, considering the early entry of polymorphonuclear neutrophils, endothelial cell activation, and the massive production of reactive oxygen species, which may further extend myocardial injury [Jordan et al., 1999; Lucchesi, 1990]. In addition, increased numbers of primed and/or activated blood leukocytes and of platelet-leukocyte aggregates have been shown to correlate with an increased risk of acute ischemic events [de Servi et al., 1991; Lindmark et al., 2001; de Servi et al., 1995; Berliner et al., 2000].

### Example 10

### Effect of CP-AP-4, CP-3(iv) and EP80317 on Infarct Size, Cardiac Troponin I and ROS Generation 48 Hours Following Transient Left Coronary Artery Ligation Surgery in CD36^{+/+} Mice

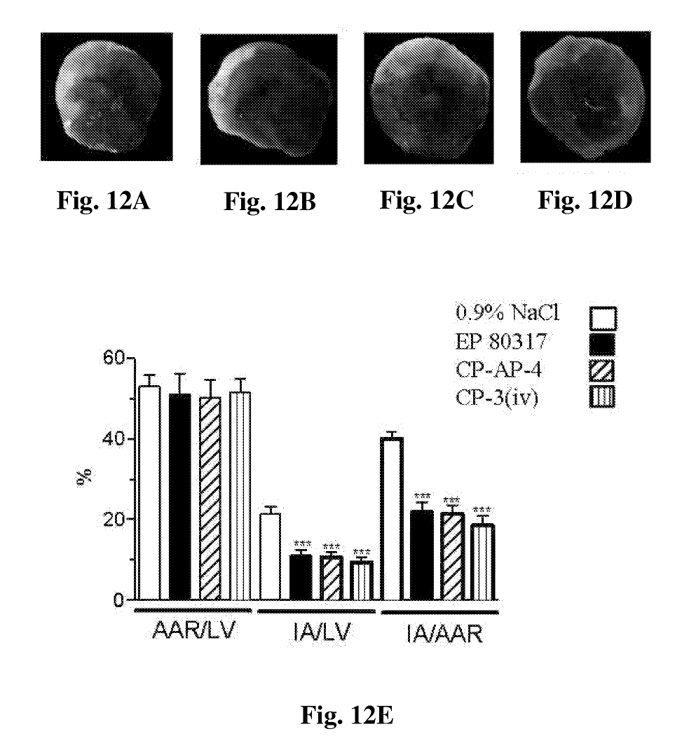

As shown in FIG. 12E, transient LCAL caused a consistently large area-at-risk that did not differ between treatment, 53 (±3), 51 (±5), 50 (±4) and 52 (±3) % in 0.9% NaCl (n=6), EP 80317 (n=6), CP-AP-4 (n=4) and CP-3(iv)-treated (n=8) mice, respectively. Infarct area (IA) and area at risk (AAR) of the left ventricle (LV), after 30-min ligation and 48 hours reperfusion are illustrated as bar graphs. The IA/LV ratios were reduced by 48, 50 and 56% (p<0.001) in mice treated with EP 80317, CP-AP-4 and CP-3(iv), respectively (FIG. 12E). Similar results were observed if the data were expressed as infarct area to AAR (IA/AAR) (FIG. 12E). These results demonstrate that azapeptide-based CD36 ligands exhibit cardioprotective effect following transient left coronary artery ligation surgery.

### Example 11

### Effect of CP-AP-4, CP-3(iv) and EP80317 on Cardiac Troponin I (cTnI) Levels 48 Hours Following Transient Left Coronary Artery Ligation Surgery in CD36^{+/+} Mice

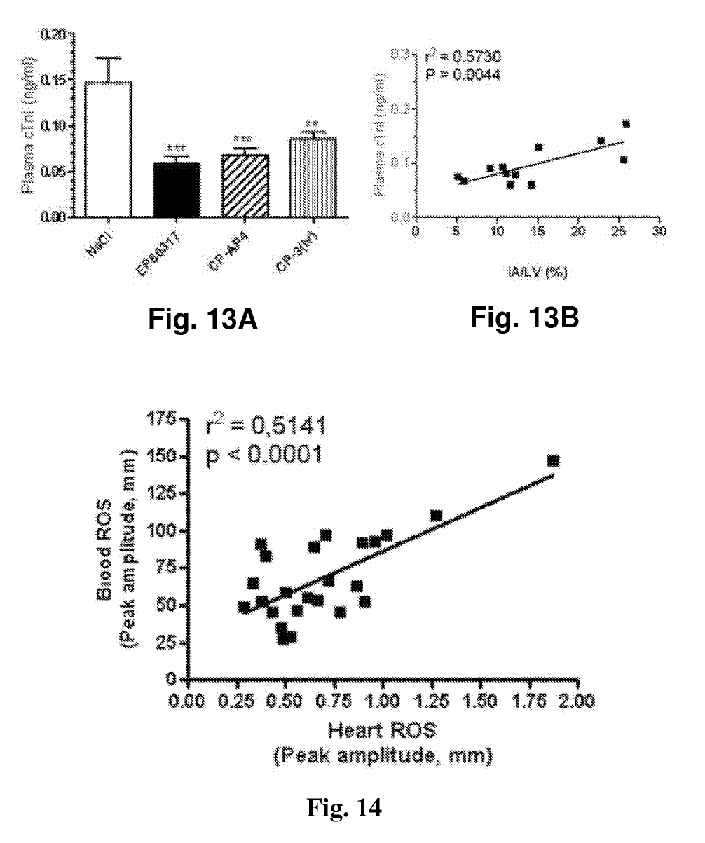

FIG. 13A demonstrates reduced cardiac troponin I (cTnI) levels in plasma of mice at 48 hours post-ischemia that were treated for 2 weeks with 289 nmol/kg of EP 80317 (n=6) (positive control) , CP-AP-4 (n=8) and CP-3(iv) (n=12) by 60%, 54% (P<0.001) and 42% (P<0.01), respectively, compared to vehicle (n=8). The plasma levels of cTnI correlated positively with the infarct area by 57% (P<0.005) (FIG. 13B). These results strongly support that the cardioprotective effect of CD36 ligands is associated with reduced myocardial necrosis in C57BU6 (CD36^{+/+}) mice.

### Example 12

### Correlation Between ROS Generation in LV Homogenate and Whole Blood at 6 Hours Post-Ischemia, in Mice Treated for 2 Weeks with Either Vehicle or 289 nmol/kg of EP 80317, CP-AP-4 and CP-3(iv)

FIG. 14 shows that there is a strong correlation (51%, p<0.0001) between blood and myocardial ROS at 6 hours post-ischemia, in mice treated or not for 2 weeks with either vehicle or 289 nmol/kg of EP 80317, CP-AP-4 and CP-3(iv). These data may suggest that activated blood leukocytes accumulating to the peri-infarct area during reperfusion may play a role in myocardial ROS generation, and are in line with reduced blood ROS and myocardial neutrophil numbers at 48 hours post-ischemia, as described in Example 9.

### Example 13

### Effect of EP 80317, CP-AP-4 and CP-3(iv) on Lactate Concentration at 6 Hours Following Transient Ischemia in C57BU6 Mice

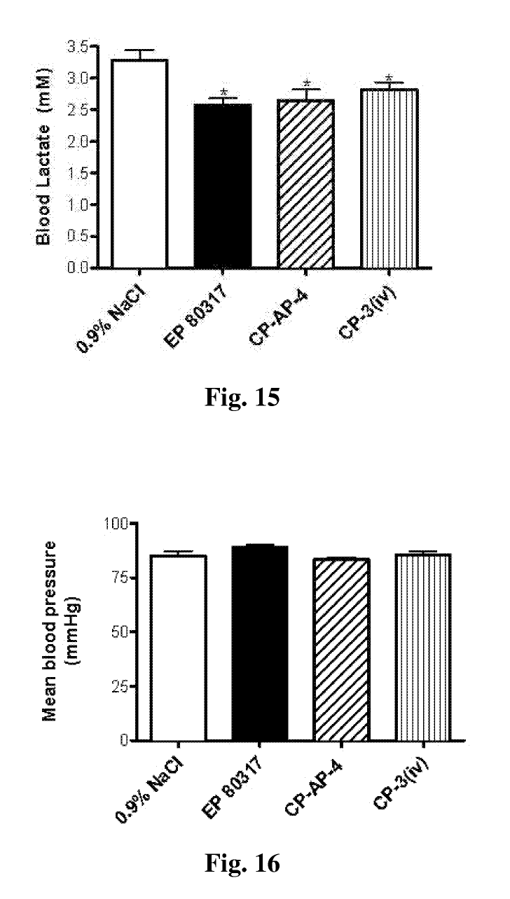

FIG. 15 shows that lactate concentration in mice following 6-hour of reperfusion are reduced by 34, 32 and 27% (P<0.05, compared to vehicle) in mice treated for 2 weeks with 289 nmol/kg of EP 80317, CP-AP-4 and CP-3(iv), respectively (n=4-6 mice per group). Blood (5 μl) was withdrawn 5 min following the 6-hour reperfusion period for lactate level determination in mice treated for 2 weeks with 289 nmol/kg of EP 80317, CP-AP-4 and CP-3(iv) by 34, 32 and 27% (P<0.05), respectively, compared to vehicle (n=4-6 mice per group). *, P<0.05 compared to 0.9% NaCl-treated C57BU6 mice.

### Example 14

### Effect of EP 80317, CP-AP-4 and CP-3(iv) on Systemic Hemodynamics at 6 Hours Following Transient Ischemia in C57BL/6 Mice

FIG. 16 shows that a 2-week treatment with 289 nmol/kg of EP 80317, CP-AP-4 and CP-3(iv) did not modify systemic hemodynamics at 6 hours following transient ischemia in C57BL/6 mice.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise. As used herein, the term “comprising” is intended to mean that the list of elements following the word “comprising” are required or mandatory but that other elements are optional and may or may not be present.

### REFERENCES

Bastie C C, Hajri T, Drover V A, Grimaldi P A, Abumrad N A (2004) CD36 in myocytes channels fatty acid to a lipase-accessible pool that is related to cell lipid and insulin responsiveness. Diabetes 53: 2209-2216

Belanger C, Elimam H, Lefebvre J, Borgeat P, Marleau S (2008) Involvement of endogenous leukotriene B4 and platelet-activating factor in polymorphonuclear leucocyte recruitment to dermal inflammatory sites in rats. Immunology 124: 295-303

Berliner S, Rogowski O, Rotstein R, Fusman R, Shapira I, Bornstein N M, Prochorov V, Roth A, Keren G, Eldor A, Zeltser D (2000) Activated polymorphonuclear leukocytes and monocytes in the peripheral blood of patients with ischemic heart and brain conditions correspond to the presence of multiple risk factors for atherothrombosis. Cardiology 94: 19-25

Bodart V, Febbraio M, Demers A, McNicoll N, Pohankova P, Perreault A, Sejlitz T, Escher E, Silverstein R L, Lamontagne D, Ong H (2002) CD36 mediates the cardiovascular action of growth hormone-releasing peptides in the heart. Circ Res 90: 844-849

Bonen A, Campbell S E, Benton C R, Chabowski A, Coort S L M, Han X-X, Koonen D P Y, Glatz J F C, Luiken J J F P (2004) Regulation of fatty acid transport by fatty acid translocase/CD36. Proc Nutr Soc 63: 245-249

Brundert, M., Moore, K. J., Merkel, M., Heeren, J., and Rinninger, F. Scavenger receptor CD36 has a role in HDL metabolism in mice. Circulation 114[11], 226. 2006.

Chabowski A, Coort S L M, Calles-Escandon J, Tandon N N, Glatz J F C, Luiken J J F P, Bonen A (2004) Insulin stimulates fatty acid transport by regulating expression of FAT/CD36 but not FABPpm. Am J Physiol Endocrinol Metab 287: E781-E789

Chabowski A, Momken I, Coort S L M, Calles-Escandon J, Tandon N N, Glatz J F C, Luiken J J F P, Bonen A (2006) Prolonged A M P K activation increases the expression of fatty acid transporters in cardiac myocytes and perfused hearts. Mol Cell Biochem published on line: DOI: 10.1007/s11010-006-9140-8

Ci X, Frisch F, Lavoie F, Germain P, Lecomte R, van Lier J E, Benard F, Carpentier A C (2006) The effect of insulin on the intracellular distribution of 14(R,S)-[18F]Fluoro-6-thia-heptadecanoic acid in rats. Mol Imaging Biol 8: 237-244

Croteau E, Benard F, Cadorette J, Gauthier M E, Aliaga A, Bentourkia M, Lecomte R (2003) Quantitative gated PET for the assessment of left ventricular function in small animals. J Nucl Med 44: 1655-1661

Crozier S J, Zhang X, Wang J, Cheung J, Kimball S R, Jefferson L S (2006) Activation of signaling pathways and regulatory mechanisms of mRNA translation following myocardial ischemia-reperfusion. J Appl Physiol 101: 576-582

Davidson S M, Hausenloy D, Duchen M R, Yellon D M (2006) Signalling via the reperfusion injury signalling kinase (RISK) pathway links closure of the mitochondrial permeability transition pore to cardioprotection. Int J Biochem Cell Biol 38: 414-419

de Servi S, Mazzone A, Ricevuti G, Mazzucchelli I, Fossati G, Gritti D, Angoli L, Specchia G (1995) Clinical and angiographic correlates of leukocyte activation in unstable angina. J Am Coll Cardiol 26: 1146-1150

de Servi S, Ricevuti G, Mazzone A, Ghio S, Zito A, Raffaghello S, Specchia G (1991) Granulocyte function in coronary artery disease. Am J Cardiol 68: 64B-68B

Dennis S C, Gevers W, Opie L H (1991) Protons in ischemia: where do they come from; where do they go to? J Mol Cell Cardiol 23: 1077-1086

Dolinsky V W, Dyck J R (2006) Role of AMP-activated protein kinase in healthy and diseased hearts. Am J Physiol Heart Circ Physiol 291: H2557-H2569

Dyck J R, Lopaschuk G D (2006) AMPK alterations in cardiac physiology and pathology: enemy or ally? J Physiol 574: 95-112

Febbraio M, Podrez E A, Smith J D, Hajjar D P, Hazen S L, Hoff H F, Sharma K, Silverstein R L (2000) Targeted disruption of the class B scavenger receptor CD36 protects against atherosclerotic lesion development in mice. J Clin Invest 105: 1049-1056

Febbraio M, Silverstein R L (2007) CD36: implications in cardiovascular disease. Int J Biochem Cell Biol 39: 2012-2030

FOLCH J, LEES M, SLOANE STANLEY G H (1957) A simple method for the isolation and purification of total lipids from animal tissues. J Biol Chem 226: 497-509

Hattori Y, Akimoto K, Nishikimi T, Matsuoka H, Kasai K (2006) Activation of AMP-activated protein kinase enhances angiotensin ii-induced proliferation in cardiac fibroblasts. Hypertension 47: 265-270

Hausenloy D J, Tsang A, Yellon D M (2005) The reperfusion injury salvage kinase pathway: a common target for both ischemic preconditioning and postconditioning. Trends Cardiovasc Med 15: 69-75

Hausenloy D J, Yellon D M (2004) New directions for protecting the heart against ischaemia-reperfusion injury: targeting the Reperfusion Injury Salvage Kinase (RISK)-pathway. Cardiovasc Res 61: 448-460

Hausenloy D J, Yellon D M (2007) Reperfusion injury salvage kinase signalling: taking a RISK for cardioprotection. Heart Fail Rev 12: 217-234

Hendrickson S C, St Louis J D, Lowe J E, Abdel-Aleem S (1997) Free fatty acid metabolism during myocardial ischemia and reperfusion. Mol Cell Biochem 166: 85-94

Hopkins T A, Dyck J R B, Lopaschuk G D (2003) AMP-activated protein kinase regulation of fatty acid oxidation in the ischaemic heart. Biochem Soc Trans 31: 207-212

Horie T, Ono K, Nagao K, Nishi H, Kinoshita M, Kawamura T, Wada H, Shimatsu A, Kita T, Hasegawa K (2008) Oxidative stress induces GLUT4 translocation by activation of PI3-K/Akt and dual AMPK kinase in cardiac myocytes. J Cell Physiol 215: 733-742

Irie H, Krukenkamp I B, Brinkmann J F F, Gaudette G R, Saltman A E, Jou W, Glatz J F C, Abumrad N A, Ibrahimi A (2003) Myocardial recovery from ischemia is impaired in CD36-null mice and restored by myocyte CD36 expression or medium-chain fatty acid. Proc Natl Acad Sci USA 100: 6819-6824

Jordan J E, Zhao Z-Q, Vinten-Johansen J (1999) The role of neutrophils in myocardial ischemia-reperfusion injury. Cardiovasc Res 43: 860-878

Kantor P F, Dyck J R, Lopaschuk G D (1999) Fatty acid oxidation in the reperfused ischemic heart. Am J Med Sci 318: 3-14

King K L, Stanley W C, Rosca M, Kerner J, Hoppel C L, Febbraio M (2007) Fatty acid oxidation in cardiac and skeletal muscle mitochondria is unaffected by deletion of CD36. Arch Biochem Biophys 467: 234-238

Koonen D P Y, Glatz J F C, Bonen A, Luiken J J F P (2005) Long-chain fatty acid uptake and FAT/CD36 translocation in heart and skeletal muscle. Bioch Biophys Acta 1736: 163-180

Kovacic S, Soltys C L, Barr A J, Shiojima I, Walsh K, Dyck J R (2003) Akt activity negatively regulates phosphorylation of AMP-activated protein kinase in the heart. J Biol Chem

Kuang M, Febbraio M, Wagg C, Lopaschuk G D, Dyck J R B (2004) Fatty acid translocase/CD36 deficiency does not energetically or functionally compromise hearts before or after ischemia. Circulation 109: 1550-1557

Kudo N, Gillespie J G, Kung L, Witters L A, Schulz R, Clanachan A S, Lopaschuk G D (1996) Characterization of 5′AMP-activated protein kinase activity in the heart and its role in inhibiting acetyl-CoA carboxylase during reperfusion following ischemia. Biochim Biophys Acta 1301: 67-75

Kurien V A, Oliver M F (1971) Free fatty acids during acute myocardial infarction. Prog Cardiovasc Dis 13: 361-373

Lazar H L (2006) Alterations in myocardial metabolism in the diabetic myocardium. Semin Thorac Cardiovasc Surg 18: 289-292

Lecomte, R., Cadorette, J., Rodrigue, S., Lapointe, D., Rouleau, D., Bentourkia, M., Yao, R., and Msaki, P. Initial results from the Sherbrooke avalanche photodiode positron tomograph. IEEE Trans Nucl Sci 43, 1952-1957. 1996.

Liem D A, Honda H M, Zhang J, Woo D, Ping P (2007) Past and present course of cardioprotection against ischemia-reperfusion injury. J Appl Physiol 103: 2129-2136

Lindmark E, Wallentin L, Siegbahn A (2001) Blood cell activation, coagulation, and inflammation in men and women with coronary artery disease. Thrombosis Res 103: 249-259

Lucchesi B R (1990) Myocardial ischemia, reperfusion and free radical injury. Am J Cardiol 65: 141-231

Luiken J J F P, Coort S L M, Koonen D P Y, van der Horst D J, Bonen A, Zorzano A, Glatz J F C (2004) Regulation of cardiac long-chain fatty acid and glucose uptake by translocation of substrate transporters. Pflugers Arch—Eur J Physiol 448: 1-15

Luiken J J F P, Coort S L M, Willems J, Coumans W A, Bonen A, Van Der Vusse G J, Glatz J F C (2003) Contraction-induced fatty acid translocase/CD36 translocation in rat cardiac myocytes is mediated through AMP-activated protein kinase signaling. Diabetes 52: 1627-1634

Luiken J J F P, Koonen D P Y, Willems J, Zorzano A, Becker C, Fisher Y, Tandon N N, Van Der Vusse G J, Bonen A, Glatz J F C (2002) Insulin stimulates long-chain fatty acid utilization by rat cardiac myocytes through cellular redistribution of FAT/CD36. Diabetes 51: 3113-3119

Marleau S, Harb D, Bujold K, Avallone R, Iken K, Wang Y, Demers A, Sirois M G, Febbraio M, Silverstein R L, Tremblay A, Ong H (2005) EP 80317, a ligand of the CD36 scavenger receptor, protects apolipoprotein E-deficient mice from developing atherosclerotic lesions. FASEB J 19: 1869-1871

Matsui T, Tao J, del Monte F, Lee K-H, Li L, Picard M, Force T L, Franke T F, Hajjar R J, Rosenzweig A (2001) Akt, activation preserves cardiac function and prevents injury after transient cardiac ischemia in vivo. Circulation 104: 330-335

Menard S L, Ci X, Frisch F, Normand-Lauziere F, Cadorette J, Ouellet R, van Lier J E, Benard F, Bentourkia M, Lecomte R, Carpentier A C (2009) Mechanism of reduced myocardial glucose utilization during acute hypertriglyceridemia in rats. Mol Imaging Biol 11: 6-14

Mueller H S, Ayres S M (1978) Metabolic response of the heart in acute myocardial infarction in man. Am J Cardiol 42: 363-371

Oliver M F (2001) Antilipolytic drugs for the prevention of primary ventricular fibrillation. Diabetes Obes Metab 3: 61-65

Opie L H (2008) Metabolic management of acute myocardial infarction comes to the fore and extends beyond control of hyperglycemia. Circulation 117: 2172-2177

Park J L, Lucchesi B R (1999) Mechanisms of myocardial reperfusion injury. Ann Thorac Surg 68: 1905-1912

Patlak C S, Blasberg R G (1985) Graphical evaluation of blood-to-brain transfer constants from multiple-time uptake data. Generalizations. J Cereb Blood Flow Metab 5: 584-590

Peart J N, Headrick J P (2008) Sustained cardioprotection: Exploring unconventional modalities. Vascul Pharmacol

Poirier P, Giles T D, Bray G A, Hong Y, Stern J S, Pi-Sunyer F X, Eckel R H (2006) Obesity and cardiovascular disease: pathophysiology, evaluation, and effect of weight loss. Arterioscler Thromb Vasc Biol 26: 968-976

Sambandam N, Lopaschuk G D (2003) AMP-activated protein kinase (AMPK) control of fatty acid and glucose metabolism in the ischemic heart. Prog Lipid Res 42: 238-256

Schwenk R W, Luiken J J, Bonen A, Glatz J F (2008) Regulation of sarcolemmal glucose and fatty acid transporters in cardiac disease. Cardiovasc Res 79: 249-258

Shiraishi I, Melendez J, Ahn Y, Skavdahl M, Murphy E, Welch S, Schaefer E, Walsh K, Rosenzweig A, Torella D, Nurzynska D, Kajstura J, Leri A, Anversa P, Sussman M A (2004) Nuclear targeting of Akt enhances kinase activity and survival of cardiomyocytes. Circ Res 94: 884-891

Soltys C L, Kovacic S, Dyck J R (2006) Activation of cardiac AMP-activated protein kinase by LKB1 expression or chemical hypoxia is blunted by increased Akt activity. Am J Physiol Heart Circ Physiol 290: H2472-H2479

Sparagna G C, Hickson-Bick D L (1999) Cardiac fatty acid metabolism and the induction of apoptosis. Am J Med Sci 318: 15-21

St Pierre J, Vohl M C, Despres J P, Gaudet D, Poirier P (2005) Genetic aspects of diabetes and its cardiovascular complications: contribution of genetics to risk assessment and clinical management. Can J Cardiol 21: 199-209

Stanley W C, Lopaschuk G D, Hall J L, McCormack J G (1997) Regulation of myocardial carbohydrate metabolism under normal and ischaemic conditions. Potential for pharmacological interventions. Cardiovasc Res 33: 243-257

Taegtmeyer H (2004) Glycogen in the heart--an expanded view. J Mol Cell Cardiol 37: 7-10

Tarnayski O, McMullen J R, Schinke M, Nie Q, Kong S, Izumo S (2004) Mouse cardiac surgery: comprehensive techniques for the generation of mouse models of human diseases and their application for genomic studies. Physiol Genomics 16: 349-360

Ussher J R, Lopaschuk G D (2008) The malonyl CoA axis as a potential target for treating ischaemic heart disease. Cardiovasc Res 79: 259-268

Venardos K M, Kaye D M (2007) Myocardial ischemia-reperfusion injury, antioxidant enzyme systems, and selenium: a review. Curr Med Chem 14: 1539-1549

Wang W, Lopaschuk G D (2007) Metabolic therapy for the treatment of ischemic heart disease: reality and expectations. Expert Rev Cardiovasc Ther 5: 1123-1134

Yang J, Sambandam N, Han X, Gross R W, Courtois M, Kovacs A, Febbraio M, Finck B N, Kelly D P (2007) CD36 deficiency rescues lipotoxic cardiomyopathy. Circ Res 100: 1208-1217.

What is claimed is:

1. A method for preventing and/or treating an ischemia-related heart condition in a subject comprising administering an effective amount of a selective CD36 ligand to said subject.

2. The method of claim 1, wherein said ischemia-related heart condition is myocardial ischemia/reperfusion (I/R).

3. The method of claim 1, wherein said method further comprises (a) decreasing plasma nonesterified free fatty acids (NEFA) levels; (b) decreasing infarct size; (c) reducing myocardial NEFA uptake; (d) decreasing myocardial oxidative metabolism; (e) decreasing myocardial blood flow; (f) increasing end-diastolic and end-systolic ventricular volumes; (g) increasing stroke volume; (h) increasing the relative ratio of phosphorylated Akt to total Akt in myocardial cells; (i) increasing the relative ratio of phosphorylated AMPK to total AMPK in myocardial cells; (j) decreasing myocardial leukocyte accumulation; (k) decreasing circulating blood leukocyte activation; (l) reducing cardiac troponin I (cTnI) levels in plasma; (m) reducing lactate concentration in blood; or (n) any combination of (a) to (m).

4. The method of claim 1, wherein said selective CD36 ligand is a peptide-like compound of general Formula I:
R⁸—X—R⁹ (I)
wherein
R⁸ is absent or is a N-terminal modification;
R⁹ is absent or is a C-terminal modification; and
X is a peptide-like domain.

5. The method of claim 4, wherein X comprises an aza-amino acid such that said peptide-like domain comprises an aza inter-amino acid linkage.

6. The method of claim 4, wherein X comprises at least one D-amino acid.

7. The method of claim 4, wherein X is a peptide-like domain of formula II:
Xaa¹-Xaa²-Xaa³-Xaa⁴-Xaa⁵-Xaa⁶ (II)
wherein
Xaa¹ is L-His, D-His, Ala, Phe, a hydrocinnamyl group, a [(2S,5S)-5-amino-1,2,3,4,6,7-hexahydro-azepino (3, 2, 1-hi)indol-4-one-2-carboxylic acid group (HAIC group), or a 2-R-(2p,5p,8p)-8-amino-7-oxo-4-thia-1-aza-bicyclo 3.4.0 nonan-2-carboxylate group (ATAB group);
Xaa² is AzaPhe, AzaTyr, D-Trp or 2MeD-Trp (a D-tryptophan residue methylated at position 2, also referred to as D-Mrp);
Xaa³ is Ala, AzaLeu, AzaPro, AzaGly or D-Lys;
Xaa⁴ is Ala, Trp, AzaTyr or AzaPhe;
Xaa⁵ is D-Phe, Ala or D-Ala; and
Xaa⁶ is Lys or Ala.

8. The method of claim 7, wherein Xaa⁴ is Trp.

9. The method of claim 7, wherein Xaa⁵ is DPhe.

10. The method of claim 7, wherein Xaa⁶ is Lys.

11. The method of claim 7, wherein X is:
(SEQ ID NO: 2)
(a) (D/L)His-AzaPhe-Ala-Ala-DPhe-Lys;
(SEQ ID NO: 3)
(b) Ala-AzaPhe-Ala-Trp-DPhe-Lys;
(SEQ ID NO: 4)
(c) His-AzaTyr-Ala-Trp-DPhe-Ala;
(SEQ ID NO: 5)
(d) Ala-AzaTyr-Ala-Trp-DPhe-Lys;
(SEQ ID NO: 6)
(e) His-DTrp-AzaLeu-Trp-Ala-Lys;
(SEQ ID NO: 7)
(f) His-DTrp-AzaLeu-Ala-DPhe-Lys;
(SEQ ID NO: 8)
(g) Phe-DTrp-Ala-AzaTyr-DPhe-Lys;
(SEQ ID NO: 9)
(h) Ala-DTrp-Ala-AzaTyr-DPhe-Lys;
(SEQ ID NO: 10)
(i) Hydrocinnamyl-DTrp-Ala-AzaTyr-DPhe-Lys;
(SEQ ID NO: 11)
(j) Ala-DTrp-azaLeu-Trp-DPhe-Lys;
(SEQ ID NO: 12)
(k) Ala-DTrp-Ala-AzaPhe-DPhe-Lys;
(SEQ ID NO: 13)
(l) His-DTrp-AzaPro-Trp-DPhe-Lys;
(SEQ ID NO: 14)
(m) His-DTrp-AzaGly-Trp-DPhe-Ala;
(SEQ ID NO: 15)
(n) HAIC-2MeDTrp-DLys-Trp-DPhe-Lys;
or
(SEQ ID NO: 16)
(o) ATAB-2MeDTrp-DLys-Trp-DPhe-Lys.

12. The method of claim 11, wherein X is Ala-AzaPhe-Ala-Trp-DPhe-Lys (SEQ ID NO:3), HAIC-2MeDTrp-DLys-Trp-DPhe-Lys (SEQ ID NO:15), Ala-DTrp-Ala-AzaPhe-DPhe-Lys (SEQ ID NO:12) or His-DTrp-AzaPro-Trp-DPhe-Lys (SEQ ID NO:13).

13. The method of claim 4, wherein R⁹ is NH₂.

14. A method for determining whether a test compound may be useful for preventing and/or treating an ischemia-related heart condition, said method comprising determining the binding of said compound to a CD36 polypeptide or a fragment thereof, wherein the binding of said compound to said CD36 polypeptide or fragment thereof is indicative that said compound may be useful for preventing and/or treating said ischemia-related heart condition.

15. The method of claim 14, wherein said ischemia-related heart condition is myocardial ischemia/reperfusion (I/R).

16. The method of claim 14, wherein said CD36 polypeptide or fragment thereof is a human CD36 polypeptide or a fragment thereof.

17. The method of claim 14, wherein said CD36 polypeptide or fragment thereof is expressed at the surface of a cell.

18. A method for determining whether a test compound may be useful for preventing and/or treating an ischemia-related heart condition, said method comprising
contacting said test compound with a cell expressing a CD36 polypeptide or a fragment thereof; and
measuring a CD36-associated activity,
wherein a modulation of said CD36-associated activity in the presence of said test compound is indicative that said test compound may be useful for preventing and/or treating said ischemia-related heart condition.

19. The method of claim 18, wherein said ischemia-related heart condition is myocardial ischemia/reperfusion (I/R) injury.

20. The method of claim 18, wherein said CD36 polypeptide or fragment thereof is a human CD36 polypeptide or a fragment thereof.
